(19) United States
(12) Patent Application Publication
(10) Pub. No.: US 2023/0377719 A1
(43) Pub. Date: Nov. 23, 2023

(54) Systems and Methods for Routing Medical Images Using Order Data

(71) Applicant: Blackford Analysis Ltd., Edinburgh (GB)

(72) Inventors: Keith HOUSTON, Birmingham (GB); David MAYNE, Edinburgh (GB)

(21) Appl. No.: 17/750,138

(22) Filed: May 20, 2022

(51) Int. Cl. G16H 30/20; G16H 10/20; G16H 50/70

(52) U.S. Cl. CPC G16H 30/20; G16H 10/20; G16H 50/70

(57) ABSTRACT

A system and method for processing medical images using order data associated with the medical images. Order data relating to a clinical request for a patient is identified. A current study of medical images can be associated with the order data. Relevancy matching rules are applied to medical image data to identify clinical software applications that are relevant for processing of the medical images. The relevancy matching rules include an order data rule that is applied to the order data associated with the medical image in order to provide additional context regarding the clinical reason for the medical images.

### FIELD

The described embodiments relate generally to processing medical image data, and in particular to processing medical image data using clinical software applications.

### INTRODUCTION

The following is not an admission that anything discussed below is part of the prior art or part of the common general knowledge of a person skilled in the art.

Electronic medical data is generated about patients during various interactions with a medical organization. Medical data is often collected when a patient is imaged using an image acquisition device. This medical data typically includes both medical image data and associated image metadata. Medical image data can be used for various clinical purposes, such as assessing, diagnosing and monitoring the progression of a disease or injury.

It is often desirable to perform image processing and/or automated analysis of medical image data before the medical images are presented to clinicians. The image processing may be intended to support a clinician's review of the scan by, for example, quantifying clinically relevant features in the images, or adding new medical images that are derived in some way from the original medical image and may provide clinically useful data.

Challenges arise in effectively routing medical data between systems that collect the medical data and clinical software applications that process and analyze the medical data. For instance, medical organizations may use multiple different information systems to collect, process, and store medical data. Ensuring that data is effectively routed between these various systems is often a complex task. The complexity and potential downside of incorrect routing is magnified due to the large file sizes of medical images and the computational resources required by clinical software applications.

A patient may visit and be imaged by an image acquisition device on multiple different occasions. In some cases, a patient may be imaged using multiple, different image acquisition devices in relation to the same clinical issue. During processing of the medical image data, it may be desirable to have a complete set of all matching studies and images to provide the most effective processing and/or analysis.

Existing medical image routing systems and workflows can encounter challenges in determining what type of processing and/or analysis should be performed on a given medical image, image series, or study. Thus, medical image data may not be routed to the optimal set of clinical software applications for processing prior to the image data (and associated processed data) being presented to a clinician. For instance, the routing of medical image data in existing systems is often not contextual and may not properly assess the relevancy of the image data or associated metadata. Assessing the relevancy of medical data may be particularly challenging when considering image data and/or image metadata that may be related across multiple studies.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In accordance with this disclosure, systems and methods are provided that can improve the identification and routing of medical image data to clinical software application. In particular, systems and methods described herein can identify related medical image data based on order data reflecting a clinical purpose or need associated with the medical image data. The identified medical image data can also be routed to specific clinical software applications that are identified, at least in part, based on the clinical purpose/need associated with the medical image data.

Order messages can be parsed to identify order data associated with particular medical image data such as a medical imaging study. The medical image data may be identified as a collection of medical imaging data that was generated in response to a corresponding order (e.g. the order data initiated the process that led to the medical imaging study being generated). The order data may represent, or at least provide an indication of, the clinical purpose underlying the imaging test that generated the corresponding medical image data. The order data can be used to populate a patient model for the patient associated with the medical image data. The patient model can be used to match a clinical software application with the medical image data. The medical image data can then be routed to the matched clinical software application for processing. Incorporating the order data representing the clinical purpose into the patient model used to match the medical image data with the clinical software application can improve the accuracy of the matching system. This can help ensure that a clinician is provided with processed medical image data that is relevant to the underlying clinical purpose for which the medical imaging was generated.

Related medical image data can also be identified using the order data contained within the patient model. The related medical image data can be retrieved and combined with the medical image data as an assembled study set. This assembled study set can be provided to the matched clinical software application for processing. This may further enhance the relevance of the processing, by providing further context to generate the processed medical image data.

In an aspect of this disclosure, there is provided a method for processing a plurality of medical images using a plurality of clinical software applications, the method comprising: receiving, at a processor, order data, the order data comprising clinical request data relating to a patient; receiving, at the processor from a first imaging device, a current study corresponding to the order data for the patient, the current study comprising current study metadata and one or more current series, each of the one or more current series comprising current series metadata; applying, at the processor, one or more relevancy matching rules associated with the plurality of clinical software applications to the current study, the one or more relevancy matching rules comprising an order data rule, wherein the order data rule is applied to the order data corresponding to the current study; based on the applying the one or more relevancy matching rules associated with the plurality of clinical software applications: determining, at the processor, that the current study is a matched current study; and identifying, at the processor, a matching clinical software application in the plurality of clinical software applications based on applying the order data rule, generating, at the processor, an assembled matched study set comprising the matched current study; retrieving, at the processor, image data corresponding to the assembled matched study set; and processing, at the processor the assembled matched study set and the image data corresponding to the assembled matched study set using the matching clinical software application to generate a processed study.

Determining, at the processor, that the current study is a matched current study can include sending, from the processor to each of a plurality of enterprise image management devices, a query for one or more candidate prior studies based on the matched current study, the one or more candidate prior studies each comprising prior study metadata and one or more prior series, each of the one or more prior series comprising prior series metadata; receiving, at the processor from at least one enterprise image management device of the plurality of enterprise image management devices, candidate prior study data relating to the one or more candidate prior studies; determining, at the processor, that the one or more candidate prior studies are one or more matched prior studies by: applying a related study order data rule of the one or more relevancy matching rules to the one or more candidate prior studies; where the assembled matched study set can include the one or more matched prior studies.

The current study can be determined to be the matched current study based on applying one or more particular matching rules of the one or more relevancy matching rules, and applying the related study order data rule can include: identifying a particular related study order data rule associated with the one or more particular matching rules; and applying the related study order data rule by applying the particular related study order data rule.

The order data can be received from an electronic medical record (EMR) system.

The order data can be received from a radiology information system (RIS) system.

The order data can be received from any other compatible system.

The order data can include at least one selected from the group of: message header data, comment data, patient identification data, patient demographic data, patient visit data, insurance data, guarantor data, patient allergy data, common order data, observation request data, diagnosis data, observation data, clinical trial identification data, and billing data.

The order data rule can define a data matching pattern that specifies a set of relevant data from one or more data fields that must be matched in order to satisfy the order data rule.

The one or more data fields can be selected from the group of: a message header data field, a comment data field, a patient identification data field, a patient demographic data field, a patient visit data field, an insurance data field, a guarantor data field, a patient allergy data field, a common order data field, an observation request data field, a diagnosis data field, an observation data field, a clinical trial identification data field, and a billing data field.

The method can include receiving at least one order message at a parser engine; and parsing, using the parser engine at the processor, the at least one order message to identify the order data comprising the clinical request data based on at least one clinical request parsing rule.

The current study data can be received at the processor directly from an image acquisition device.

Each relevancy matching rule can specify one or more corresponding patient model fields and the method can include applying each relevancy matching rule by: determining the presence or absence of relevant data within the one or more corresponding patient model fields of the patient model associated with the current study; and determining that the current study is matched according to that relevancy matching rule in response to determining the presence of the relevant data within the one or more corresponding patient model field.

For at least one of the relevancy matching rules, the at least one patient model field to be evaluated can be selected from the group of: an order notes field, a diagnosis description field, a diagnosis code field, a procedure code field, and a study indication field.

The processed study can be provided in a DICOM format.

The processed study can include at least one of processed patient metadata, processed imaging data and processed order data.

The matched clinical application can automatically process the assembled matched study set.

The current study data can be defined using a DICOM data format.

The order data can be defined using an HL7 format or a FHIR format.

In accordance with an aspect of this disclosure, there is provided a system for processing a plurality of medical images using a plurality of clinical software applications, the system comprising: a memory, wherein one or more relevancy matching rules are stored in the memory, the one or more relevancy matching rules associated with the plurality of clinical software applications, the one or more relevancy matching rules comprising an order data rule; a network device; a processor in communication with the memory and the network device, the processor configured to: receive, using the network device, order data, the order data comprising clinical request data relating to a patient; receive, using the network device from a first imaging device, a current study corresponding to the order data for the patient, the current study comprising current study metadata and one or more current series, each of the one or more current series comprising current series metadata; apply the one or more relevancy matching rules to the current study, where the order data rule is applied to the order data corresponding to the current study; based on the applying the one or more relevancy matching rules: determine that the current study is a matched current study; and identify a matching clinical software application in the plurality of clinical software applications based on applying the order data rule; generate an assembled matched study set comprising the matched current study; retrieve, using the network device, image data corresponding to the assembled matched study set; and process the assembled matched study set and the image data corresponding to the assembled matched study set using the matching clinical software application to generate a processed study.

The processor can be configured to determine that the current study is a matched current study by: sending, using the network device to each of a plurality of enterprise image management devices, a query for one or more candidate prior studies based on the matched current study, the one or more candidate prior studies each including prior study metadata and one or more prior series, each of the one or more prior series including prior series metadata; receiving, using the network device from at least one enterprise image management device of the plurality of enterprise image management devices, candidate prior study data relating to the one or more candidate prior studies; determining that the one or more candidate prior studies are one or more matched prior studies by: applying a related study order data rule of the one or more relevancy matching rules to the one or more candidate prior studies; wherein the assembled matched study set is generated to include the one or more matched prior studies.

The processor can be configured to: determine that the current study is the matched current study based on applying one or more particular matching rules of the one or more relevancy matching rules; and apply the related study order data rule by: identifying a particular related study order data rule associated with the one or more particular matching rules; and applying the related study order data rule by applying the particular related study order data rule.

The order data can be received from an EMR system.

The order data can be received from an RIS system.

The order data can be received from any other compatible system.

The order data can include at least one selected from the group of: message header data, comment data, patient identification data, patient demographic data, patient visit data, insurance data, guarantor data, patient allergy data, common order data, observation request data, diagnosis data, observation data, clinical trial identification data, and billing data.

The order data rule can define a data matching pattern that specifies a set of relevant data from one or more data fields that must be matched in order to satisfy the order data rule.

The one or more data fields can be selected from the group of: a message header data field, a comment data field, a patient identification data field, a patient demographic data field, a patient visit data field, an insurance data field, a guarantor data field, a patient allergy data field, a common order data field, an observation request data field, a diagnosis data field, an observation data field, a clinical trial identification data field, and a billing data field.

The processor can be configured to: receive at least one order message at a parser engine; and parse, using the parser engine, the at least one order message to identify the order data comprising the clinical request data based on at least one clinical request parsing rule.

The current study can be received using the network device directly from an image acquisition device.

Each relevancy matching rule can specify one or more corresponding patient model fields and the processor can be configured to apply each relevancy matching rules by determining the presence or absence of relevant data within the one or more corresponding patient model fields of the patient model associated with the current study; and determining that the current study is matched according to that relevancy matching rule in response to determining the presence of the relevant data within the one or more corresponding patient model fields.

For at least one of the relevancy matching rules, the at least one patient model field to be evaluated can be selected from the group of: an order notes field, a diagnosis description field, a diagnosis code field, a procedure code field, and a study indication field.

The processed study can be provided in a DICOM format.

The processed study can include at least one of processed patient metadata, processed imaging data and processed order data.

The matched clinical application can be configured to automatically process the assembled matched study set.

The current study data can be defined using a DICOM data format.

The order data can be defined using an HL7 format or a FHIR format.

In accordance with an aspect of this disclosure, there is provided a non-transitory computer-readable medium with instructions stored thereon for processing a plurality of medical images using a clinical software application, that when executed by a processor, performs the method as described herein.

It will be appreciated by a person skilled in the art that a system, device, method or computer program product disclosed herein may embody any one or more of the features contained herein and that the features may be used in any particular combination or sub-combination. Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a system diagram of a medical organization including a medical data processing system.

FIG. 2 is a block diagram of an example data processing device that may be used in the medical data processing system of FIG. 1.

FIG. 3 is a block diagram of a data processing flow sequence that may be implemented in the medical data processing system of FIG. 1.

FIG. 4 is a flowchart illustrating an example method for processing medical image data.

FIG. 5 is a flowchart illustrating an example method for parsing order messages.

FIG. 6 is screenshot of an example of a configuration file that may be used to parser order messages.

FIG. 7 is a screenshot of pseudo-code that may be used to define an example relevancy matching rule.

FIG. 8 is screenshot of an example order message that may be received by the medical data processing system of FIG. 1.

### DESCRIPTION OF EXEMPLARY EMBODIMENTS

Various apparatuses or methods will be described below to provide an example of an embodiment of the claimed subject matter. No embodiment described below limits any claimed subject matter and any claimed subject matter may cover methods or apparatuses that differ from those described below. The claimed subject matter is not limited to apparatuses or methods having all of the features of any one apparatus or methods described below or to features common to multiple or all of the apparatuses or methods described below. It is possible that an apparatus or methods described below is not an embodiment that is recited in any claimed subject matter. Any subject matter disclosed in an apparatus or methods described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal or a mechanical element depending on the particular context. Furthermore, the term “communicative coupling” indicates that an element or device can electrically, optically, or wirelessly send data to another element or device as well as receive data from another element or device.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g. 112a, or 112₁). Multiple elements herein may be identified by part numbers that share a base number in common and that differ by their suffixes (e.g. 112₁, 112₂, and 1123). All elements with a common base number may be referred to collectively or generically using the base number without a suffix (e.g. 112).

The embodiments of the systems and methods described herein may be implemented in hardware or software, or a combination of both. In some cases, the example embodiments described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g. a keyboard, mouse, a touchscreen, and the like), and at least one output device (e.g. a display screen, a printer, a wireless radio, and the like) depending on the nature of the device. For example and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high level procedural, declarative, functional or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible.

As used herein, the term “DICOM” refers to the Digital Imaging and Communications in Medicine (DICOM) standard for the communication and management of medical imaging information and related data as published by the National Electrical Manufacturers Association (NEMA).

As described herein, “HL7” refers to the Health Level 7 (HL7) standard as published by Health Level Seven International.

As used herein, the term “patient” generally refers to any human or animal or other subject that may undergo an imaging test to acquire medical image data from that human or animal. The systems and methods described herein may also be applied to “imaging phantoms” during various calibration and/or testing of image acquisition devices.

As described herein, “medical images”, “image data”, or “images” refers to image data collected by image acquisition devices. The images are visual representations of an area of body anatomy that may be used for various purposes such as clinical analysis, diagnosis and/or medical interventions, commonly referred to as radiology. These images can include images of removed organs and tissues and/or images of an area of anatomy captured in-situ. Medical imaging can also establish a database of normal anatomy and physiology to make it possible to identify abnormalities.

Medical images can be acquired using various imaging technologies including X-ray Plain Film (PF), digital X-rays, Cardiology imaging devices including a cardiology PACS server, Computed Tomography (CT) images, ultrasound images, nuclear medicine imaging including Positron-Emission Tomography (PET), Veterinary imaging devices, Magnetic Resonance Imaging (MRI) images, mammographic images, or any other standardized images used in a medical organization. The medical images may be collected using analog means such as film and then subsequently scanned, or may be constructed from data originally collected using digital sensor means such as a Charge Coupled Device (CCD) and processed into image data. The image data may be provided in JPEG, JFIF, JPEG2000, Exif, GIF, BMP, PNG, PPM, PGM, PBM, PNM, WebP, HDR, HEIF, or any other known format. The image data may be provided in an uncompressed image format, in a lossless compressed format, or in a lossy compressed format.

A patient accessing healthcare with a medical organization can have various interactions with clinicians and various investigations and tests in order to diagnose, assess and monitor the state or progression of a condition such as a disease or injury. Through these investigations, medical data is generated and collected related to the patient and the condition or conditions being evaluated. The medical data associated with a particular patient may be referred to herein as patient medical data. This medical data is stored and managed using one or more electronic data management systems such as an electronic medical record (EMR) system, a radiology information system (RIS), a picture archiving and communication system (PACS) and so on.

In many cases, an imaging test is ordered as part of a patient's interaction with the medical organization. Medical imaging data is collected about a patient when they are imaged by an image acquisition device during the imaging test. This medical imaging data generally includes image data (also referred to as image pixel data or pixel data) and associated image acquisition metadata.

Medical images can be generated using various different types of image acquisition devices. A “modality” refers to the categorical type of image acquisition generated by different image acquisition devices (e.g. CT, MR, x-ray, ultrasound or PET; as in, “those images have modality ‘CT”’). The different modalities or types of image acquisition devices refer to the technology used to create the image.

Medical imaging data is generated when a patient undergoes a medical imaging test using a specific imaging modality. The medical imaging data output from an individual imaging test is referred to as a study or imaging study. Characteristics of an imaging study such as format, size and number of images generated by an individual imaging test (also referred to as a scan or a scanning episode) can vary based on numerous factors such as the imaging modality type, the clinical purpose for the imaging test (e.g. the diagnostic or clinical need for the imaging test), the part of the body (the area of anatomy) being scanned etc.

An individual imaging test can capture a single image, a series of images of a single area of anatomy, multiple series of images covering a single area of anatomy, multiple series of images covering multiple different areas of anatomy, a video of an area of anatomy etc. An individual imaging test may capture multiple series of images in various circumstances, such as when the image acquisition device is operable in multiple modes of operation (e.g. an MR scanner) or to acquire images before and after administration of a contrast agent for example. In some cases, an imaging test may acquire medical imaging data in the form of video imaging data (e.g. a video of a beating heart). In many cases, video imaging data can be treated as a series of images for the purposes of data processing and/or analysis.

A patient may undergo multiple imaging tests in the course of seeking medical treatment. This can include scans of the same or different anatomy areas using the same or different image acquisition devices with each imaging test. In some cases, a patient may undergo a planned sequence of scans of the same area of anatomy using the same image acquisition device to monitor a disease and/or to track treatment. This may be referred to as a longitudinal sequence of studies.

The medical imaging data generated by the imaging tests can be stored by the medical organization (also referred to as a medical provider) using one or more electronic data management systems (e.g. PACS, RIS etc.). However, managing and correlating the medical imaging data from different imaging tests is often challenging. For instance, many medical providers use multiple different electronic management systems to store and manage medical data including medical imaging data. Between the different electronic management systems, patient medical data may not be correctly correlated or associated. In some cases, patient medical data may not even be correlated between different studies stored in the same electronic management system.

It is often desirable to perform additional processing and/or analysis on medical imaging data before the medical imaging data is presented to a clinician. Such processing and/or analysis is typically intended to support a clinician's review of the scan by, for example, quantifying clinically relevant features in the images, or adding new, clinically useful, image data derived in some way from the originals. However, determining the appropriate processing and/or analysis to perform on a given imaging study is a complex challenge. Images of the same part of a patient's anatomy may be acquired for many different clinical purposes. The type of processing and/or analysis that would be most useful to a clinician can vary depending on the part of the patient's anatomy imaged as well as the clinical purpose of the imaging test.

Medical organizations often experience challenges routing medical imaging data to clinical applications used for processing or analysis. In many cases, medical imaging data must be manually routed to the clinical applications. This results in time wasted for the technicians who perform the routing and introduces the possibility of human error. However, existing automated systems and methods for routing medical imaging data to clinical applications are complex and often result in sub-optimal data routing.

One approach to automated image data routing is to forward all studies of a particular body part to a given clinical application. However, this approach can result in redundant processing and the generation of superfluous information. For example, an MR-Head study is an example of a study of medical image data collected from a magnetic resonance imaging procedure conducted on a patient's brain. Using a body-part based approach might send every MR-Head study to a clinical application that analyses the image data for stroke symptoms. However, many MR-Head studies are ordered for purposes other than stroke identification (e.g. detection of multiple sclerosis lesions). This can result in superfluous clinical information generated by the clinical application (e.g. for MR-Head studies unrelated to stroke identification), and an increase in resource utilization and processing costs. In addition, many MR-Head studies may be ordered for other clinical purposes that are not captured by the body-part specific routing rules. This may require further manual routing and processing, which can again occupy unnecessary technician time and re-introduce the possibility of human error.

Clinical software applications that perform image processing and/or analysis are often computationally expensive. Medical images also tend to be large in size. This increases the computational expense associated with processing and/or analysis operations. The large size of medical images can also introduce challenges in managing network bandwidth when transferring images between devices for storage, processing, analysis, and review. Optimizing the processing performed by the clinical software applications can reduce computational loads and minimize computational and network resource consumption.

As described herein, clinical software applications are software applications that generate additional information about a medical study or studies through the analysis of image data (including pixel data) and/or image metadata of one or more images of the study or studies. Such analysis might range from simple to highly complex calculations including statistical inferences and machine learning. The analysis by the clinical software application may be “global” in the sense that it takes account of pixels and/or meta-data across an entire matched set of images, or several sets of images. Alternatively, the analysis may be a “local” analysis that, e.g. only performs an analysis based on a single image or fragment of meta-data. The clinical software application analysis may add clinically relevant information as described herein. Further, the clinical software application may add information, including probabilistic information such as confidence levels, probabilities, and/or certainties. Alternatively or in addition, the clinical software application may generate new medical image data based on transformations, enhancements, or other operations on the original image data.

Clinical software applications produce result data, i.e. new information in addition to the original image data. The result data can take various forms, such as one or more new images or image regions, modified images or image regions, and/or additional metadata derived from the original images. Examples of image processing and analysis that may be performed by a clinical software application include image compression, data encryption, information enhancement, image registration, anatomy recognition, contrast detection, image quality, quantification of brain abnormalities and changes from MR images in order to track disease evolution, liver iron concentration analysis from MR images of the liver, and the removal of personally identifying information among many others.

Processing and analyzing related studies together can reduce the cumulative processing required to process each individual study (for example, a region of interest identified in a first study that forms part of a longitudinal sequence of studies may be used with a second study in the sequence to analyze changes in the region of interest). Thus, it is often desirable to identify related relevant studies, series, or image data which may be desirable for analysis alongside the current study.

In medical data processing systems, the image data and image metadata associated with an imaging study is typically stored in a DICOM format. Accordingly, automated routing systems may analyze the DICOM data (both imaging data and metadata) associated with a study. This DICOM data associated with a particular study, in some cases combined with manual input from a clinician or technician, can be used to identify a clinical application to which the medical imaging data can be routed. In some cases, the DICOM data may also be used to identify medical imaging data of related imaging studies.

The DICOM image metadata typically includes information about how the medical image data was acquired, such as scanner data, anatomy data (e.g. the body regions imaged), image spatial data (e.g. spatial information about pixels and slice spacings), study identifier data, image identifier data, acquisition date, acquisition time etc. However, the DICOM data does not always provide contextual information relating to the medical imaging data. For instance, the DICOM data may not indicate why the medical imaging test (that generated the medical imaging data) was ordered or why the medical imaging data is being requested by a clinician. As a result, the DICOM data may be insufficient to determine the type of processing and/or analysis that should be applied to the medical imaging data in order to provide a clinician with a useful set of processed medical imaging data.

The DICOM data may also be insufficient to identify related studies that are generated through different imaging tests. As a result, medical data processing systems that rely solely or primarily on DICOM data associated with a medical imaging study may produce sub-optimal results in terms of routing the medical image data to clinical software applications and/or initiating processing by the clinical software applications.

In some cases, non-imaging DICOM messages such as DICOM worklist messages may be used to route the medical image data to clinical software applications and/or initiate processing by the clinical software applications. For instance, DICOM worklist messages can be used to convey textual scan- and/or order-related data that provide information to a technologist and/or radiologist to enable them to select a patient to scan next, how that patient should be scanned, and/or what subsequent processing operations should be carried out on the generated medical image. Typically, DICOM worklist messages are received or retrieved from a Radiology Information System, or RIS. Other non-imaging DICOM messages can be used to communicate non-imaging data (i.e. data unrelated to the scheduling or carrying out of medical imaging tests) such as lab results data, electrocardiogram data, and/or structured report data. These non-imaging DICOM messages can be received from a PACS or other DICOM archive, a medical device that generates non-imaging data, or a clinical application for example.

These non-imaging DICOM messages can be evaluated to for various purposes, including identifying new medical image data (e.g. a new study), identifying medical image data related to a current study (e.g. one or more prior studies), identifying clinical software applications that match a current study, triggering fetching of new medical image data, triggering pre-fetching of one or more prior studies, triggering processing of one or more current studies using a clinical software application etc. This may provide further context than is possible through analysis of the DICOM imaging data alone.

The present disclosure provides systems and methods for processing a plurality of medical images using a plurality of clinical software applications. Order data can be identified that is related to medical imaging data. The order data can include clinical request data that represents a clinical request for the patient (e.g. a request relating to the medical imaging data and/or the associated imaging test). The medical imaging data can be matched with, and routed to, a particular clinical application based, at least in part, on analysis of the order data. Medical imaging data (e.g. new studies and/or related studies) may also be identified based on analysis of order data.

The order data may reflect a clinical purpose associated with the medical imaging data. The clinical purpose generally refers to the reason for which the medical imaging data was generated—i.e. the medical investigation that is the underlying purpose behind the clinician ordering the imaging test that generated the medical imaging data. Using the order data to match the medical imaging data with a particular clinical application can ensure that the medical imaging data is processed in a manner relevant to the underlying clinical purpose.

The order data can be used to populate an instance of a patient model for the patient for whom the medical image data was collected. The medical data processing system can define or store a patient model that defines a data structure for storing data relating to one or more patients. The patient model can specify a plurality of patient model fields. Each patient model field can specify a type of data relating to a patient that can be stored for that patient within an instance of the patient model.

When data is collected about a particular patient (e.g. through their interactions with the medical organization), the data can be stored in an instance of the patient model that is tied to that particular patient. That is, each patient can have an associated patient model instance that contains various different types of data relating to that patient and their interactions with the medical organization. Each patient model instance can collect and store data for the corresponding patient in accordance with the data structure defined by the patient model. Each patient model instance can include patient identification data usable to uniquely identify that patient (e.g. an accession number, a medical record number, or demographic data usable to uniquely identify the patient).

The patient model instances can be stored in a patient model cache in the medical data processing system. When data relating to a patient is received by the medical data processing system, the data may be used to update the corresponding patient model instance. When performing various data management and/or data processing operations (e.g. routing medical imaging data to a clinical software application), the medical data processing system can use the data stored in the patient model instances in the patient model cache in order to perform data processing operations relating to data for the corresponding patient.

The patient model instance for a patient can be used to match a clinical software application with the medical image data for that patient. The medical image data can then be routed to the matched clinical software application for processing. The patient model instance can include the order data extracted from order messages in addition to other data related to the same patient, such as various imaging studies for that patient or order data extracted from other order messages relating to the same patient.

Order data may be identified from order messages received by the medical data processing system. Order messages can be analyzed to identify data fields expected to contain order data with information relevant to the processing of an associated medical imaging data. For example, the order data fields may include data indicative of a clinical purpose associated with an imaging test (and the associated medical imaging data). The order data fields can include various user-configurable fields such as a procedure code field, a study indication field, a diagnosis description field, a diagnosis code field or an order notes fields for example.

An order message can be parsed to extract order data from the identified order data fields. The extracted order data can then be used to populate the patient model instance for the corresponding patient. The order data incorporated into the patient model instance can be used to route medical imaging data (relating to that patient) to one or more clinical software applications. This may allow the medical imaging data to be processed and/or analyzed in a more contextual manner, thereby providing a clinician with more relevant processed image data.

The order data can also be analyzed to identify medical imaging data available to be routed to one or more clinical software applications. For example, the order data may be used to identify new medical imaging data that can be matched with a clinical software application. This may help identify new medical imaging data that is ready to be processed but has not yet been retrieved by the processing system. Accordingly, the order data can be used to trigger fetching of the new medical imaging data.

The order data provided to the patient model instance can also be used to identify related medical imaging data that may be useful during processing and/or analysis of medical imaging data (for that patient), such as other related medical imaging data generated for the same patient for the same clinical purpose. This may allow the medical imaging data to be processed and/or analyzed in a more efficient manner, by ensuring that related medical imaging data is identified. Accordingly, the order data can be used to trigger fetching of the related medical imaging data (in some cases, even before the new medical imaging data has been retrieved or completely retrieved).

The term “order messages” generally refers to messages received by the medical data processing system in a format (typically a non-DICOM format) used to transmit orders for a given medical organization. The particular format used to convey order data may vary between medical organizations. For example, the order messages may be HL7 messages or FHIR messages received by the medical data processing system. For ease of exposition, the present application has been described using the example of HL7 messages. However, it should be understood that the systems and methods described herein can also be applied to other types of order messages including FHIR messages for example.

The order messages can be evaluated to identify order data that can be used for various purposes, including identifying new medical image data (e.g. a new study), identifying medical image data related to a current study (e.g. one or more prior studies), identifying clinical software applications that match a current study, triggering fetching of new medical image data, triggering pre-fetching of one or more prior studies, triggering processing of one or more current studies using a clinical software application etc. The order data can be added to a patient model instance that can be used for the various purposes noted above.

The incoming HL7 messages can include a stream of HL7 messages from various locations, such as an electronic healthcare record (EMR) system (e.g. an EPIC system) and/or a radiological information system (RIS). Each HL7 message may relate to a portion of a patient's interaction with the medical organization.

Each patient may be assigned a unique patient identifier (e.g. a medical record number or an accession number) during their interactions with the medical organization. The medical data processing system can use the unique patient identifier to associate the order message data with a particular patient. This unique patient identifier can be used to generate a patient model instance for the particular patient. The unique patient identifier can also be used to associate DICOM data for the same patient with the corresponding patient model instance.

Alternatively, patient-related data can be used to associate the order message data with a particular patient. For instance, patient demographic data such as name, age and sex can be used to associate the order message data with the particular patient. Various techniques can be used to match patient-related data received at different times, including fuzzy matching techniques in some cases. This may be particularly useful where a patient is erroneously assigned more than one unique patient identifier or where there are slight variations in data entry.

Incoming HL7 messages such as order messages and/or order update messages can be parsed to identify order data. In particular the incoming HL7 messages may be parsed to identify order data associated with medical imaging data that needs to be processed (referred to herein as a current study) before being displayed to a clinician.

Each incoming HL7 message may be parsed to determine whether the message contains order data. The order data may be subsequently analyzed to determine whether it is associated with the medical imaging data of a current study. Relevancy matching rules can be applied to the order data to identify a clinical software application that matches the corresponding current study and/or to identify one or more additional studies (e.g. prior studies) that may be relevant to the corresponding current study. The relevancy matching rules may be applied to a patient model instance containing the order data.

The medical data processing system can include a parser configured to parse incoming order messages. The parser can be configured to parse order messages (e.g. HL7 messages) received by the medical data processing system. The parser can be configured to parse and extract relevant data from the order messages that can be used to populate a patient model instance. DICOM data received by the medical data processing system can be combined with the data extracted from the order messages to update the patient model instance. When a current study is received by the medical data processing system (typically subsequent to receiving the order messages), the medical data processing system can determine that the current study is related to a particular patient model instance (e.g. to order data or DICOM data contained in that patient model instance). The patient model instance can then be used to determine whether the current study is relevant to a particular clinical software application.

The order messages may be parsed using a parser configuration file. The parser configuration file can define a plurality of message parsing rules. The parser can be configured to parse the order messages received by the medical data processing system according to the message parsing rules defined in the parser configuration file. For instance, the parser configuration file can specify particular fields in each order message that are expected to contain relevant order data. The parser can be configured to extract the order data from the specified fields. The parser configuration file can also specify how the extracted data from the fields of the HL7 messages are used to generate a patient model instance (i.e. defining the patient model fields into which specific kinds of extracted data should be added).

The parser configuration file can also specify message accept/reject rules. The message accept/reject rules can specify which HL7 messages are to be analyzed by the parser in order to generate a patient model. The parser may apply the message accept/reject rules to determine whether an HL7 message will be parsed to extract order data. The parsed HL7 data can be used, along with DICOM data associated with the same patient, to generate a more accurate patient model instance that includes contextual data relating to the clinical purpose for the patient's interaction with the medical organization.

The medical data processing system can include a relevancy engine that can be configured to determine one or more clinical applications that match a current study. The relevancy engine can be used to automatically route the current study to the matched clinical software application(s).

The relevancy engine can match a current study to a clinical application using clinical application relevancy matching rules. The clinical application relevancy matching rules can be applied to the patient model instance of the patient associated with the current study to determine whether the current study matches a corresponding clinical software application. The clinical application relevancy matching rules may be predefined for each clinical software application.

Alternatively, the clinical application relevancy matching rules may be defined by a routing model (e.g. a trained machine learning model). The routing model can receive patient model instance data related to the current study as an input. The patient model instance data can be received from a patient model instance stored for the patient corresponding to the current study.

The patient model instance data can include medical image data and image metadata from the current study. This medical image data and image metadata may be defined using the DICOM format. The data defined using the DICOM format may be referred to as DICOM data.

The patient model instance data can also include order data associated with the patient (for whom the current study was carried out). The order data may be identified through analysis of order messages received by the medical data processing system.

The routing model can output a matched application output. The matched application output can identify one or more clinical software applications that are matched to the current study. The matched application output can indicate which clinical software application(s) the current study should be routed to for processing and/or analysis. The medical data processing system can use the matched application output to automatically route the current study to the one or more matched clinical software applications.

Various different methods for training the routing model can be applied. A supervised learning method can involve a user (e.g. a clinician or technician) manually selecting the matched clinical software application(s) for a training study. The manually selected matched clinical software application(s) can be used as a label for the current study data (e.g. DICOM data, order data etc.) associated with the current study that would be used as an input to the routing model. This process can be repeated for multiple current studies.

An optimization algorithm can be applied to identify a routing pattern using positive reinforcement data (where a study is matched to a particular clinical application) and negative reinforcement data (where a study is not matched to a particular clinical application). The routing patterns identified through the optimization algorithm can subsequently be applied by the routing model to automatically match a current study with one or more clinical software applications. This may eliminate the need for a user to manually select the matched clinical software application(s).

A supervised training approach may be particularly useful because of the sometimes fluid nature of HL7 data. HL7 data can be organized using different schemas for different medical organizations. For example, the precise segment, name and field number used for order data and order-related data may vary across institutions. Accordingly, the routing patterns may differ between medical organizations. Applying the supervised training method may provide a medical organization with flexibility to train the routing model based on the existing workflows developed within that organization.

Alternatively or in addition, the routing model may be trained using unsupervised learning methods. For example, the routing model may be trained using a historical study routing dataset that identifies how prior studies were routed to matched clinical software applications. Optionally, the routing model once trained may undergo further supervision and training on the part of a user to ensure sufficient accuracy in the routing model.

Optionally, the medical data processing system may store a parser model that can be used to generate a dynamic parser configuration file. The parser model may be a machine learning model trained to identify and extract relevant data from HL7 messages that can be used to route medical imaging data to relevant clinical software applications. The parser model can receive feedback from the relevancy engine indicating the specific patient model fields used to match a current study to a corresponding clinical software application (as well as those fields that were not used to match the current study). The parser model can use the feedback data to identify which fields in the patient model, and corresponding order messages, are most relevant for matching the current study to the clinical software application. The parser model can then be used to update the parser configuration file to prioritize extraction of the more relevant order data fields.

The medical data processing system can use the data extracted from the HL7 messages to route the processed medical imaging data (received from a clinical software application). This may ensure that the processed medical imaging data is routed to the appropriate reporting platform or system.

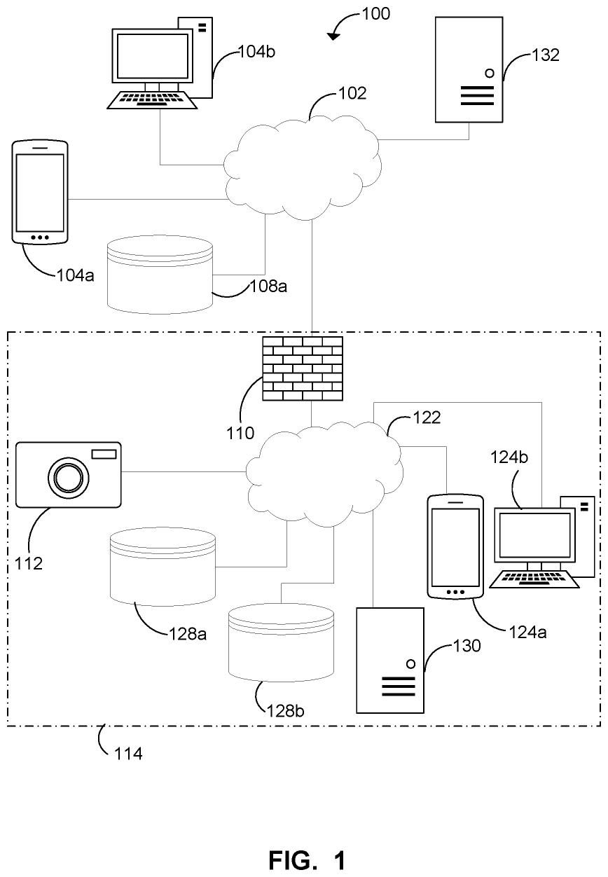

Reference is first made to FIG. 1, which illustrates an example medical data processing system 100. The example system 100 includes a plurality of user devices 104 such as mobile device 104a and computer device 104b, a network 102, a server 132, and an imaging device in the form of an enterprise image management device 108a.

As illustrated, system 100 includes a medical organization 114. Medical organization 114 has an associated medical organization network 122 that is connected to network 102 by a firewall 110. A plurality of medical organization user devices 124 (e.g. mobile device 124a and computer device 124b) may operate within the medical organization network 122. The medical organization user devices 124 and the plurality of user devices 104 can generally be provided using various types of computing devices, as discussed below. The medical organization network 122 can also include a server 130 (e.g. a processing server), imaging devices in the form of one or more enterprise image management devices 128a and 128b and imaging devices in the form of one or more image acquisition devices 112. The server 132 and enterprise image management device 108a may be referred to, respectively, as a remote server 132 and a remote enterprise image management device 108a in the sense that they are external to the medical organization network 122.

Alternatively, the processing server 130 may be external to the medical organization 114 and the enterprise image management devices 128 may forward image data and associated metadata to the external processing server via network 122, firewall 110, and network 102.

User devices 104 and medical organization user devices 124 may be used by end users to access an application (not shown) running on remote server 132 over network 102 and network 122. For example, the application may be a web application, or a client/server application. The user devices 104 and medical user devices 124 may be desktop computers, mobile devices, or laptop computers. The user devices 104 and medical user devices 124 may display the web application, and may allow a user to review medical data, including medical images and image metadata, from medical organization 114. A user of user devices 104 and medical organization devices 124 may be a clinician user at medical organization 114 who may review the medical data, including processed medical data from the clinical software applications. A clinician user may be a radiologist whose role is to review (or read) medical images, or a referring clinician (for example, the non-radiologist clinician who referred the patient for a scan) who may receive a report from the radiologist.

The users at user devices 104 and medical organization devices 124 may include an administrator user who may administer the configuration of clinical software applications or matching rules for medical organization 114.

Enterprise image management devices may include both remote enterprise image management device 108a, first enterprise image management device 128a inside the medical organization 114, and second enterprise image management device 128b inside the medical organization 114. While a single remote enterprise image management device 108a is shown, it is understood that there may be a plurality of remote enterprise image management devices. Similarly, while only two enterprise image management device 128a and 128b are shown, it is understood that there may be more enterprise image management devices.

The enterprise image management devices may be a Picture Archiving and Communication System (PACS) server, a Modality Worklist (MWL), a medical image data archive or another enterprise image management device. For example, an enterprise image management device may be an IntelePACS® from Intelerad®, an IntelliSpace® PACS from Philips®, an enterprise image management device such as the Enterprise Imaging Solution® suite from Change Healthcare®, a Medicor® MiPACS® Modality Worklist or an IBM iConnect Enterprise Archive.

Remote enterprise image management device 108a may be located remotely from the medical organization 114. There may be one or more remote enterprise image management devices 108a. For example, a remote PACS may be at an affiliated medical organization to the medical organization 114, for example, a satellite clinic. A remote enterprise image management device 108a may provide economical storage and convenient access to medical images and image metadata from multiple image acquisition devices external to medical organization 114. A PACS may support live Query/Retrieve, archive Query/Retrieve, be configured to auto-forward, or a combination of these roles.

Remote enterprise image management device 108a may be a Modality Worklist (MWL), where the MWL makes patient demographic information from a Radiology Information System (RIS) available at an image acquisition device, and providing, amongst other things, a worklist of patients who will attend the image acquisition device for imaging in the near future. The MWL may further provide in-progress studies and completed studies.

Remote enterprise image management device 108a may be a remote imaging image acquisition device configured using auto-forward to the medical organization 114.

The remote enterprise image management device 108a may store image metadata in a DICOM format, an HL7 format, an XML-based format, or any other format for exchanging image data and associated metadata.

Remote server 132 may be a commercial off-the-shelf server. Alternatively, the remote server 132 may be a server running on Amazon® Web Services (AWS®) or another similar hosting service. The remote server 132 may be a physical server, or may be a virtual server running on a shared host. The remote server 132 may have an application server, a web server, a database server, or a combination thereof. The application server may be one such as Apache Tomcat, etc. as is known. The web server may be a web server for static web assets, such as Apache® HTTP Server, etc. as is known. The database server may store user information including structured data sets, electronic form mappings, and other electronic form information. The database server may be a Structured Query Language (SQL) such as PostgreSQL® or MySQL® or a not only SQL (NoSQL) database such as MongoDB®.

The remote server 132 is in communication with processing server 130, and may provide data analysis for the processing server, or data aggregation in conjunction with processing server 130. The remote server 132 may provide a web-based interface for browsing analysis of the medical data and the clinical software applications running at processing server 130.

Alternatively, the remote server 132 may be provided inside the medical organization 114 and connected to the processing server 130 via network 122.

Alternatively, there may be more than one server providing the functionality of remote server 132, including one inside the medical organization 114 in communication with processing server 130 via network 122, and a second remote server accessible to the medical organization 114 via network 122, firewall 110, and network 102.

Network 102 may be a communication network such as the Internet, a Wide-Area Network (WAN), a Local-Area Network (LAN), or another type of network. Network 102 may include a point-to-point connection, or another communications connection between two nodes.

Network 122 may be a communication network such as an enterprise intranet, a Wide-Area Network (WAN), a Local-Area Network (LAN), or another type of network. Network 122 may include a point-to-point connection, or another communications connection between two nodes. The network 122 may exist at a single geographical location, or may span multiple geographical locations. Network 122 is separated from network 102 by firewall 110, which may provide for network address translation for the medical organization 114, virtual private network access for the remote user devices 104, access control lists for network traffic sent between network 122 and network 102 (and vice-versa).

Medical organization 114 may include one or more related medical organizations that share medical image data and associated metadata over one or more networks. The one or more related medical organizations may include one or more image acquisition devices, one or more geographical locations, a plurality of clinician users, a plurality of administrative users, and a plurality of patients attending the one or more image acquisition devices for medical imaging services.

The medical organization 114 has a firewall 110, one or more image acquisition devices 112, a first enterprise image management device 128a, a second enterprise image management device 128b, a processing server 130, one or more medical user devices 124 such as mobile device 124a and computer device 124b, and medical network 122.

Firewall 110 may be a commercial off-the-shelf network firewall that performs network translation and routing between network 102 and network 122. For example, firewall 110 may be a Cisco® firewall, a Sonicwall® firewall, or another firewall as is known.

The one or more image acquisition devices 112 may include a variety of different imaging modalities that generate medical images such as X-ray Plain Film (PF) devices, digital X-ray devices, Computed Tomography (CT) devices, ultrasound devices, nuclear medicine imaging devices including Positron-Emission Tomography (PET) devices, Magnetic Resonance Imaging (MRI) devices, mammographic devices, or any other imaging modality used in a medical organization. The one or more image acquisition devices 112 may include mobile imaging devices such as mobile CT scanners. The medical images generated by the one or more imaging devices may be collected using analog means such as film and then subsequently scanned, or may initially be collected using digital sensor means such as a Charge Coupled Device (CCD). The one or more image acquisition devices 112 may operate to produce studies of patients of the medical organization 114. The one or more image acquisition devices 112 may collect various metadata at the time it captures images of the patient. The metadata collected by the one or more image acquisition devices 112 may be in DICOM format, HL7 format, or other formats for image data and associated metadata formats as are known. The one or more image acquisition devices 112 may include, for example, a General Electric® (GE®) Revolution Apex® CT image acquisition device, a Siemens® Magnetom Vida® MR image acquisition device, and a Canon® UltiMax® x-ray image acquisition device.

The first enterprise image management device 128a and the second enterprise image management device 128b may be PACS. Alternatively, either the first or the second enterprise image management devices 128 may be a Modality Worklist at one of the one or more image acquisition devices 112. The enterprise image management device 128 may store medical image data collected at the one or more image acquisition devices 112, and image metadata corresponding to the medical image data.

The image data generated by the one or more image acquisition devices 112 and stored in the first enterprise image management device 128a and second enterprise image management device 128b may be provided in JPEG, lossless JPEG, Run-Length Encoding (RLE), JFIF, JPEG2000, Exif, GIF, BMP, PNG, PPM, PGM, PBM, PNM, WebP, HDR, HEIF, or any other known image format.

Processing server 130 may be a commercial off-the-shelf server system. The processing server 130 may be configured to execute a software platform that manages the processing of medical images, and further executes one or more clinical software applications to process the medical images, as discussed herein. The processing server 130 may be a physical server, or a virtual server running on a shared host. The processing server 130 can be a single server, or multiple different individual servers configured to work cooperatively to provide the platform application and the one or more clinical software applications. The processing server 130 is connected via network 122 to the enterprise image management devices 128, the one or more image acquisition devices 112, and medical user devices 124. The processing server 130 is further in network communication with the remote enterprise image management device 108a and remote server 132 via network 122, firewall 110, and network 102.

User devices, such as the processing server 130, can be implemented using a combination of hardware and software components. For instance, devices such as the processing server 130 can include a network unit, an output device such as a display or speakers, an input unit such as mouse or keyboard, a processor (also referred to as a processing unit), a memory, and a power unit.

The network unit may be a standard network adapter such as an Ethernet or 802.11x adapter. The memory unit can include both volatile memory and non-volatile storage memory. The power unit can provide power to the processing server 130. Processing server 130 can include an I/O unit that provides access to server devices including disks and peripherals. The I/O hardware can provide local storage access to the programs running on processing server 130.

The processor unit may include a standard or special-purpose processor. Alternatively or in addition, a plurality of processors can be used by the processor unit and may function in parallel. Alternatively or in addition, a plurality of processors can be used by the processor unit including a Central Processing Unit (CPU) and a Graphics Processing Unit (GPU). There may be a plurality of CPUs and a plurality of GPUs.

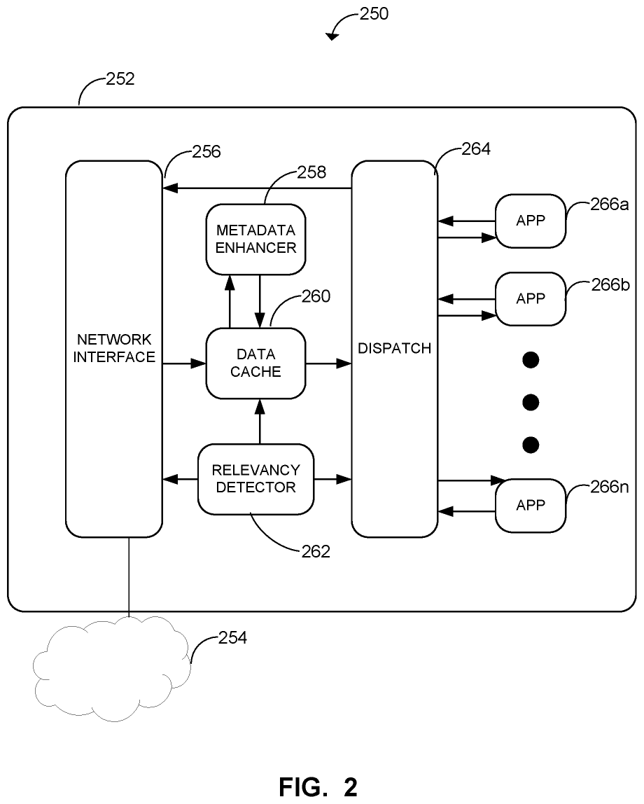

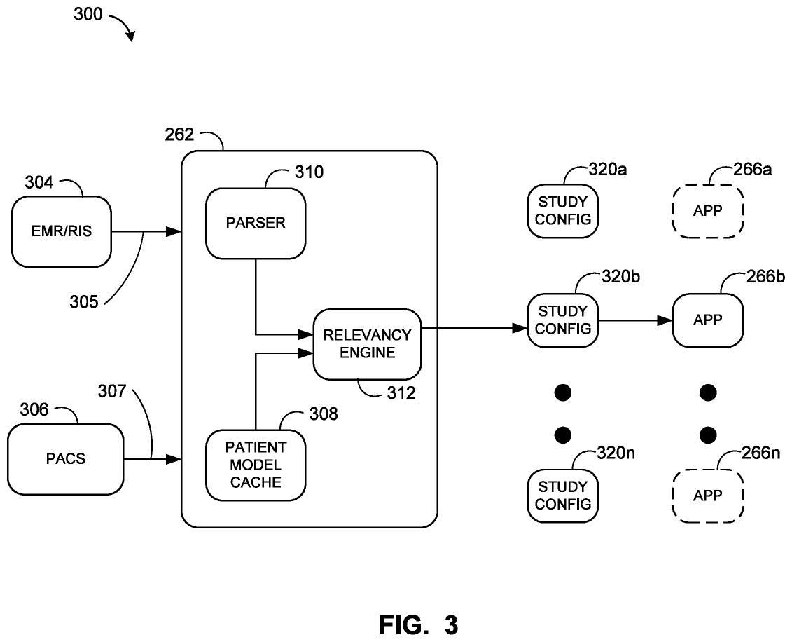

The processor unit can also execute a user interface engine that is used to generate various GUIs. The GUIs can be presented to users of the user devices to allow the users to review data such as medical images, input clinical requests and/or reports, and interact with data generated by the image processing system. The user interface engine can provide clinical software application configuration layouts to allow users to configure clinical software applications, rule configuration layouts for users to configure relevancy matching rules, parser configuration layouts to allow users to define a parser configuration file and so on. The information submitted using these interfaces may be processed by a relevancy engine and/or clinical software applications (see e.g. parser 310 in FIG. 3, relevancy engine 312 in FIG. 3 and applications 266 in FIGS. 2 and 3). The user interface engine may be an Application Programming Interface (API) or a Web-based application that is accessible via the network unit or a graphical user interface application accessed via remote desktop or a similar type of user interface engine.

The memory unit can store instructions executable by the processor to implement an operating system and one or more applications. The memory unit can store instructions for an operating system, various programs such as a platform core, a metadata enhancement engine (see e.g. metadata enhancer 258 shown in FIG. 2), a relevancy engine (see e.g. relevancy engine 312 shown in FIG. 3), a dispatcher (see e.g. dispatcher 264 shown in FIG. 2), and one or more clinical software applications (see e.g. applications 266 shown in FIGS. 2 and 3).

The operating system may be a Microsoft Windows Server operating system, or a Linux-based operating system, or another operating system.

The programs comprise program code that, when executed, configures the processor unit to operate in a particular manner to implement various functions and tools for the processing server.

The platform core provides functionality for managing the processing server. This may include providing an API available via the network unit for administration of the processing server, including configuration of the clinical software applications and relevancy engine. Alternatively, a user may access a web interface of the platform core via the user interface engine and/or the network unit to administer the processing server.

The platform core may be deployed to a physical server or a virtual server on the same network as its host medical organization. For example, the platform core may be deployed as a Windows® Service. The platform core may include a settings editor that allows an administrator to configure the platform core, or other components.

The memory unit can also store data usable by the applications operating on the device. For example, the memory unit can store one or more databases and/or caches that can be used for long-term (or at least non-volatile) storage of data such as patient model instance data, study data, image data, series data, metadata, patient data, order data etc. The databases can also store reference data accessible by components of the imaging processing system such as a parser or relevancy matching engine. For example, the reference data may include a configuration file, a configuration model, one or more matching rules etc.

Reference is next made to FIG. 2, showing an example block diagram 250 of a medical data processing system 252. Medical data processing system 252 is an example medical data processing system that may be used as a medical data processing system such as processing server 130 in system 100. As illustrated, the medical data processing system 252 is connected to a network 254.

The processing system 252 may provide an image processing platform for processing medical images and image metadata. In the example illustrated, the image processing platform includes a network interface 256 in communication with network 254, a meta-data enhancer 258, a data cache 260, a relevancy detector 262, a dispatcher 264, and one or more clinical software applications 266.

The processing system 252 can receive medical image data and associated image metadata from devices on network 254 using network interface 256. Studies received at the platform may be referred to as current studies. The current studies are sent by an enterprise image management device, for example a PACS server or a MWL server. The platform 252 may process incoming current studies by way of “jobs”. The jobs may be scheduled or driven by discrete events in the lifecycle of the associated study. For example, if a current study is scheduled via MWL, a new job may be created and started. As part of this job, the platform 252 may discover prior studies (by querying one or more medical devices), determine the relevance of received prior studies using relevancy rules, and fetch related images corresponding to the prior metadata. The current study and its prior studies are scheduled for processing at the clinical software application(s) 266.

Alternatively, when a current study is being collected at an image acquisition device and the enterprise image management device (or PACS server) and the platform 252 start receiving image data, a new job is started. As part of this job, the platform 252 may discover new data, determine the relevance of the new data, including new images and prior studies, and fetch any missing prior data. For example, the platform 252 may discover new data through parsing of order data received by the platform 252. When the current study finishes receiving image data, the platform 252 can fetch any missing current and/or prior data at high priority. Optionally, the platform 252 can pre-fetch prior study data based on the analysis of the order data (e.g. before the current study finishes receiving image data, and in some cases even before receiving any image data relating to the current study). Once all data has been fetched, the current study and its prior studies can be scheduled for processing at the clinical software application(s) 266.

The network interface 256 connects the processing server to the medical organization networks using a variety of protocols and modes of operation. The network interface 256 may receive medical image data and associated image metadata using a push mode of operation, where image devices send data to the processing server. Alternatively or in addition, the processing system 252 may actively fetch medical image data and associated metadata from imaging devices and/or storage components. The network interface 256 may send a polling request to one or more imaging devices or image storage components using a “pull” mode of operation of the medical image data and associated metadata. The network interface 256 may receive medical images and image metadata using both a pull and a push mode of operation from the image devices.

The processing system 252 may actively fetch medical image data and associated metadata based on order data received by the processing system 252. For example, the processing system 252 may parse an incoming message (e.g. an HL7 message) to identify order data. Optionally, the processing system 252 may be configured to intercept messages generated within the medical organization 114 that may contain order data (e.g. HL7 messages transmitted through the medical organization network 122). The processing system 252 can analyze the order data and determine that medical image data and/or associated metadata should be fetched (e.g. in response to determining that new medical image data is available and/or in response to determining that related medical image data is available).

The data cache 260 of the processing system 252 is a software component that can be used to assemble received image data. The data cache 260 may cache both medical images and image metadata.

The data cache 260 can be configured to storage image data and/or associated metadata locally within the platform 252. Once the image data and/or associated metadata is stored locally by the data cache 260, the components of the platform 252 retrieve can image data and associated metadata from the data cache 260 without the need to transmit data through the network interface 256 and through network 254.

For example, data cache 260 may store image data and/or associated metadata following a first query sent by the platform 252. When a second query is transmitted by a component of the platform 252, the data cache 260 can provide a locally cached copy of the image data and/or associated metadata to the requesting component. In this manner, the data cache 260 may provide image data and associated metadata to the components of the platform 252 without the need for a query request to be sent using network interface 256 and network 254.

The data cache 260 can allow incoming studies (and images associated with studies) to arrive at different times. The data cache 260 may buffer incoming studies. In some cases, the data cache 260 may hold partial study data received for an incoming study until all of the data for the study is received. The data cache 260 may identify the held study as being in a pending stage until all of the data for the study is received.

The cache can include a storage portion in memory where medical images, image metadata, study data, study metadata, series data, and series metadata may be stored as the processing server is generating an assembled matched study set for processing at a clinical software application. Study data, study metadata, series data, series metadata, image data, and image metadata may be received by the processing server 252 at the network interface 256 and then stored in the cache 260.

The cache 260 may hold image data and associated metadata until all relevant data (e.g. as determined by relevancy engine 312) is available for processing or analysis by an application 266. Alternatively or in addition, the data cache 260 may cache all received data for a specified caching period. As a result, any study, series or image data need only be fetched once over the network 254 within the specified caching period. This may allow the cached data to be used by several different clinical software applications without requiring separate data transmissions over network 254.

Optionally, data cache 260 may include two or more distinct caches. For example, cache 260 may include a first cache for image data and a second cache for metadata associated with the image data. This may simplify data management, as the image data may tend to be large in size while the associated metadata may be smaller in size. Accordingly, different data management protocols may be applied to the data stored in the image cache as opposed to the data stored in the metadata cache.

The cache 260 or another storage medium (e.g. a database) at the processing server 250 can be configured to store metadata using a metadata object model. The metadata object model can include various different types of metadata objects, referred to as metadata object instances. Each metadata object instance can be associated with a different type of information. For instance, the metadata object model can include patient model instances (including patient metadata associated with a particular patient), study instances (including study metadata associated with a particular study), series instances (including series metadata associated with a particular series), and image instances (including image metadata associated with a particular image instance).

Optionally, a metadata object instance may be a composite instance that includes one or more other instances as a portion of the metadata object instance (e.g. a patient instance may include one or more related study instances, a study instance may include one or more related series instances, and a series instance may include one or more related image instances). The composite instance may be defined based on a relationship between the various instances. The relationship may be a hierarchical relationship (e.g. a patient instance can include one or more studies instances, each study instance includes one or more series instances, and each series instance includes one or more image instances).

Each metadata object instance can include one or more metadata elements. The metadata elements may vary depending on the type of metadata object instance. The metadata elements can be separated into fields for the corresponding metadata object instance.

The metadata object model can be normalized such that metadata object instances incorporating metadata from various different systems connected to the processing server 250 are stored in an analogous manner. For instance, each metadata object instance may include the same set of metadata fields (or at least each metadata object instance of the same instance type may include the same set of metadata fields).

Each instance can be associated with a corresponding unique entity identifier. The unique entity identifier may be defined to be globally unique across the entire medical organization network 122. For example, an instance may have a corresponding unique entity identifier that is defined using DICOM metadata. The unique entity identifier may be globally unique across the entire DICOM environment.

The data cache 260 may be implemented using an off-the-shelf caching program such as memcached, redis, or another caching application as is known. The data cache 260 may also provide for read through and lazy loading functionality such that data is only loaded into the data cache 260 as needed, e.g. upon request of other components of the processing system 252.

The metadata enhancer 258 can enhance the image metadata in the data cache 260 by adding new or additional metadata that may enhance understanding of the existing image data or metadata. The additional metadata may be determined by analysis of the original image and/or associated image metadata. Alternatively or in addition, the metadata enhancer 258 may enhance image metadata by adding new metadata that may be determined by analysis of pixel data in the data cache 260.

The metadata enhancer 258 can be configured to analyze incoming studies and images to generate supplementary image metadata. The supplementary image metadata may be generated as a metadata object and stored in association with a current study (e.g. in data cache 260 and/or in persistent storage provided by an enterprise image management device 128). Alternatively or in addition, the metadata enhancer 258 may receive processed studies (following processing by a clinical software application 266) from the dispatcher 264, and may generate metadata objects corresponding to the processed studies and/or processed images. The generated metadata objects may be stored in association with the current study.

The relevancy detector 262 determines if a received study matches one or more of the clinical software applications 266 based on one or more relevancy matching rules associated with the configuration of each clinical software application. The relevancy detector 262 may further determine that, based on the relevancy rules used to match the current study, that additional queries should be made to determine the existence of one or more matching prior studies, and/or one or more matching prior series. The determination for further queries to locate prior studies or prior series may be made based on a rule configuration (e.g. based on the particular rule that was used to match the current study). To locate the matching prior studies and series, the relevancy detector may send queries using network interface 256 to one or more enterprise image management devices. An example relevancy detector 262 is described in further detail herein below with reference to FIG. 3.

The dispatcher 264 determines an assembled study set based on the received query response data from the relevancy detector 262 and/or the data cache 260. The dispatcher 264 may receive study data, image data, series data, and metadata from the query response or from the data cache 260. The dispatcher 264 may determine the assembled data set based on relevant data and is responsible for loading, notifying, or executing any clinical software applications 266 matching the current study based on the rules configured in the clinical software application configuration. Any matching clinical software applications 266 may then be executed to process the assembled data set. The dispatcher 264 may further send the processing result of a clinical software application using network interface 256, so that it may be sent to one or more enterprise image management devices. The processing result may include new medical images, or new image metadata, and the processing result may be associated with the studies, series, and images associated with the assembled data set.

The one or more clinical software applications 266 provide data processing capabilities to the assembled result set. Clinical software applications that process medical data automatically can create processed data and other medical data, including medical images and metadata. Examples of clinical software applications include an image registration application, a registration application, an anatomy recognition application, a contrast detection application, an image quality application, a Personal Health Information (PHI) Removal application, a quantification application (e.g. of brain abnormalities and/or changes between studies and/or an estimate of liver-iron-concentration from a study of the liver) and so forth.

Dispatcher 264 dispatches the assembled matched study set to the one or more clinical software applications (for example, clinical software application 266a-266n). This may involve an inter-process communication, or a network communication using network unit 256. Once processing is completed, the dispatcher may receive a processed study based on the assembled matched study set and may send the processed study to one or more image devices in the plurality of image devices (for example, enterprise image management device 128a, enterprise image management device 128b in FIG. 1A) using network unit 204.

Each of the clinical software applications 266a-266n is a software application that accepts the assembled matched study set and performs some processing on the data contained in the assembled matched study set and then generates a processed study that is sent to the dispatcher 264. While only three clinical software applications are shown in FIG. 2, it is understood that there may be any number of clinical software applications, and potentially many more clinical software applications.

The clinical software applications may run in a process at processing server 252. Alternatively, the clinical software applications may run in a virtual machine (e.g. using VMware ESX) or a container (e.g. using Docker) at processing server 252. Alternatively, the clinical software applications may be located separately from processing server 252 in network communication with processing server 252, and in such a case the dispatcher 264 may transmit the assembled matched study set to the clinical software application using network unit 256, and receive the processed study back from the clinical software application using network unit 256. Alternatively, a combination of clinical software application hosting may be used, for example a first clinical software application in one or more clinical software applications may run in a process at a processing server 252, a second clinical software application in the one or more clinical software applications may be located separately from the processing server 252, and a third clinical software application in the one or more clinical software applications may be located in a virtual machine or container at processing server 252.

Alternatively, the clinical software applications may be software modules provided via a remote cloud-hosted service which are accessed by using a remote API.

Referring now to FIG. 3, shown therein is a block diagram illustrating an example data processing flow sequence 300 that may be implemented using a processing system, such as processing system 250 for example. The example data processing flow sequence in FIG. 3 is shown for an example implementation of a relevancy detector 262.

The relevancy detector 262 can be configured to receive data from various devices and systems connected to the processing system 250. For instance, the relevancy detector 262 may receive data through a network interface such as network unit 256 described herein above.

As shown in FIG. 3, the relevancy detector 262 can receive various types of data from enterprise image management device 306 such as a PACS or a Modality Worklist server. The relevancy detector 262 can receive image data and/or associated metadata from the enterprise image management devices 306. The relevancy detector 262 can receive various types of metadata, including study metadata, series metadata, and image metadata from the enterprise image management device 306. The metadata is typically received using a standardized format, such as the DICOM format.

The relevancy detector 262 can also receive various types of data from medical data management system 304. The medical data management system 304 can include various types of data management systems used to manage medical data such as electronic healthcare record (EMR) systems and radiological information systems (RIS). The data received from the medical data management systems 304 is also typically received using a standardized format (often a non-DICOM format) such as the HL7 format or FHIR format.

The relevancy detector 262 can receive various messages from the medical data management system 304. For instance, the relevancy detector 262 can be configured to accept unsolicited HL7 or FHIR messages from the medical data management system 304. That is, the relevancy detector 262 can receive HL7 messages from the medical data management system 304 without initiating specific requests for messages.

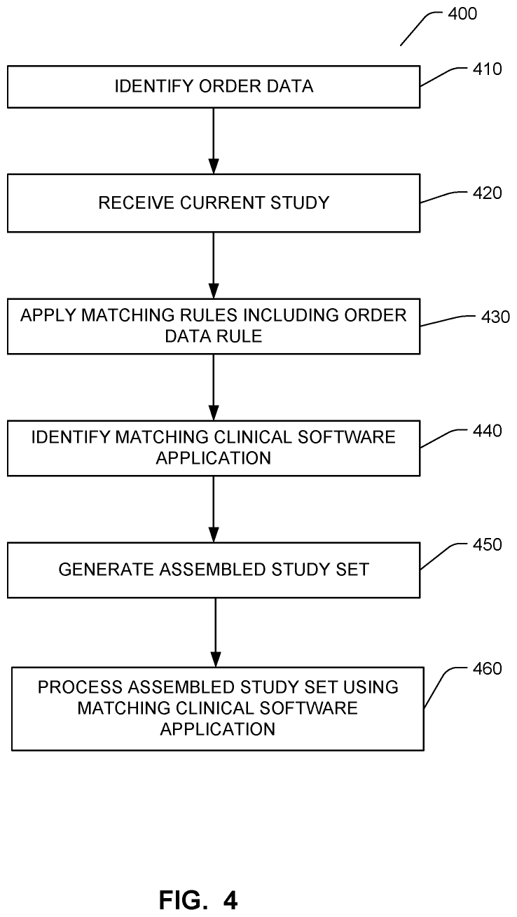

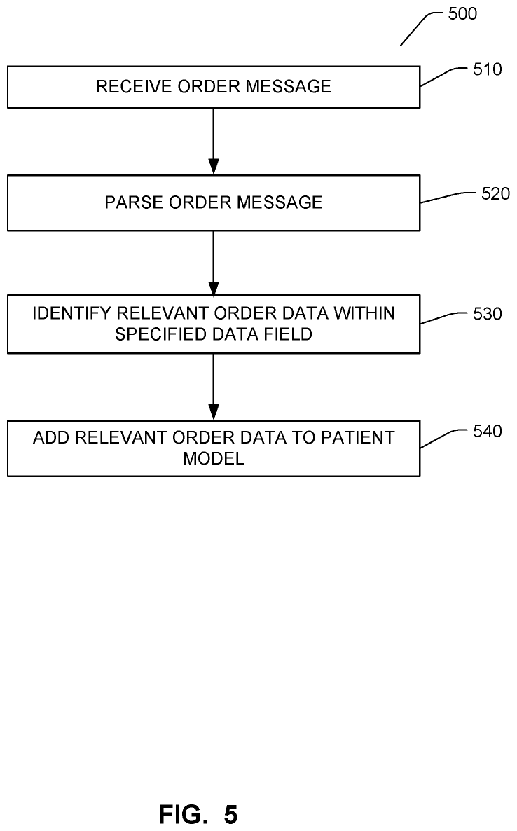

The data received by the relevancy detector 262 can be used for various purposes. The relevancy detector 262 can identify order data in the messages received from the medical data management system 304. The order data can be used as part of various processes, such as processes for identifying relevant study data (including identifying new studies and/or relevant prior studies), processes for fetching study data (including fetching of new current study data and/or prior study data), and processes for routing study data to one or more clinical applications. Examples of methods that may be implemented using the relevancy detector 262 are described in further detail herein below with references to FIGS. 4 and 5.

As shown in the example of FIG. 3, the relevancy detector 262 can include a patient model cache 308, a parser 310, and a relevancy engine 312. The parser 310 can be configured to analyze received order messages to identify relevant order data relating to a patient. This order data can be provided to the patient model cache 308 to be included in a patient model instance for the corresponding patient. The relevancy engine 312 can then analyze the patient model instance for a given patient to identify a matching clinical software application for a current study associated with the patient model.

The patient model cache 308 can record and cache patient model instances associated with a particular patient interacting with the medical organization. The patient model cache 308 can store a plurality of patient model instances within the processing system 250. The patient model instances stored within the patient model cache 308 may be accessible to the relevancy engine 312 without the need for a query request to be sent using network interface 256.

The patient model cache 308 may hold patient model instance data for a given patient until all relevant data (e.g. as determined by relevancy engine 312) related to that patient is available for processing or analysis by an application 266. Alternatively or in addition, the patient model cache 308 may cache all patient model instance data for a specified caching period.

Each patient model instances can be a composite instance that includes one or more other instance. For instance, a patient model instance may have one or more associated study instances. The one or more associated study instances can include a current study to be processed. Each study may have one or more associated series instance. Each series may have one or more associated image instances.

Each patient model instance can include a plurality of patient metadata elements. The patient metadata elements can include a patient name, a unique patient identifier (e.g. an accession number), a patient birth date, a patient sex, comments associated with the patient, and extracted order data associated with the patient. The patient metadata elements may be defined manually by a user and/or automatically by components of the system 100, such as the enterprise image management device, PACS, MWL server, or image acquisition device. The patient metadata elements may be stored in the patient model instance using patient model fields that are defined by a global patient model.

Each patient model instance can be generated using non-DICOM data (e.g. HL7 or FHIR data) received from the medical data management system(s) 304. Furthermore, DICOM data received for the same patient (e.g. from PACS 306) can be included in the patient entity.

The parser 310 can be configured to analyze and parse message data received from the medical data management system 304. The parser 310 can intercept each message (e.g. each HL7 message) sent to the relevancy detector 262. The parser 310 can then identify order data within each message that may be relevant to one or more processes performed by the relevancy engine 312.

The parser 310 can be configured to analyze and parse message data according to a configuration file defined for the processing system 250. A single configuration file can be defined for each parser 310 implemented within a medical organization 114. The configuration file can define a set of parsing rules that can be used to identify relevant messages, identify potentially relevant order data fields within the messages, and identify and extract the order data from the corresponding order data fields. The rules defined within a given configuration file may be tailored for a given medical organization 114, e.g. to reflect the HL7 schema implemented by that medical organization 114.

Alternatively or in addition, machine learning techniques may be used to define or update the parsing rules applied by the parser 310.

The parser 310 can be configured to identify order data within the message data received from the medical data management system 304. Optionally, the extracted order data can be provided to the patient model instance for the patient associated with the order data. This can be used to update the corresponding patient model instance with additional order data relating to the patient's interaction(s) with the medical organization 114.

The parser 310 can extract patient data (e.g. order data associated with the patient) and study data (e.g. order data associated with a particular study) from the HL7 messages received from the medical data management system(s) 304. The patient data and study data can be used to populate the patient model instance for the corresponding patient that is stored in the patient model cache 308.

The parser 310 can be configured to determine whether a message contains potentially relevant order data fields. Potentially relevant order data fields can be identified as fields within the message that can be used to transmit information relating to an order. In some cases, the potential order data field may be identified based on the type of message received from the medical data management system 304. For instance, the potentially relevant order data fields may be identified based on the HL7 schema implemented by the medical organization 114.

Messages (or message types) that include potentially relevant order data fields may be accepted by the parser 310 for further processing. Optionally, the parser 310 may reject messages (or message types) that do not include potential order data fields. The parser 310 can be configured to only accept incoming messages of a message type known to contain order-specific data (e.g. ORU or ORM HL7 messages). Incoming messages of other message types may be rejected (e.g. ADT or ACK HL7 messages). The specific message types that may be accepted or rejected can be defined based on the messaging schema implemented by a particular medical organization.

The parser 310 can also be configured to reject malformed incoming messages.

The parser 310 can be configured to parse each incoming message to identify user-configurable fields in the incoming messages. The user-configurable fields may be identified as potential order data fields. Examples of user-configurable fields include a procedure code field, a study indication field, an order notes field, diagnosis code, and a diagnosis description field for example. The parser 310 can then extract the order data contained within the potential order data fields.

The parser 310 can then evaluate the potential order data fields within a message to identify the corresponding order data. The parser 310 can then extract the order data. The extracted order data can be used to populate a patient model instance in patient model cache 308 for the corresponding patient. For instance, the extracted order data can be associated with a corresponding patient model field. The extracted order data can then be stored in the corresponding patient model field in the patient model instance stored in patient model cache 308 for the corresponding patient. An example process of parsing order messages that may be performed by the parser 310 is described herein below with reference to method 500 shown in FIG. 5.

The extracted order data can also be provided to the relevancy engine 312. The relevancy engine 312 can use the extracted order data when applying relevancy matching rules to match a study with one or more prior studies and/or match a study with one or more clinical software applications. The relevancy engine 312 may also use the extracted order data to trigger matching and processing of a current study. In some cases, the relevancy engine 312 may analyze the extracted order data after it is stored in the patient model instance for the corresponding patient.

In addition to the extracted order data, the relevancy engine 312 can receive additional data that can be analyzed using the relevancy matching rules. For instance, the relevancy engine 312 can receive study metadata, series metadata, and image metadata, along with images corresponding to the study metadata, images corresponding to the series metadata, and images corresponding to the image metadata. Study metadata may refer to series metadata and image metadata. The study metadata, series metadata, and image metadata (and associated images) may also be retrieved from the patient model instance for the corresponding patient.

The relevancy engine 312 can be configured to apply a plurality of relevancy matching rules. The relevancy matching rules can be applied to a current study to identify related studies and/or identify matching clinical software applications. That is, the relevancy matching rules can be applied to the order data and the metadata associated with the current study. The relevancy engine 312 can apply the relevancy matching rules to the data stored in the patient model instance for the patient associated with the current study.

The plurality of relevancy matching rules can include one or more clinical application relevancy matching rules. Each clinical application relevancy matching rule can be used to identify a clinical software application that matches a current study to be processed. The matched clinical software application can be used to perform processing of that current study.

The plurality of relevancy matching rules can include one or more related study relevancy matching rules. Each related study relevancy matching rule can be used to identify a related study (e.g. a prior study) that matches a current study to be processed. The matched related study can be provided to the clinical software application along with the current study to support the processing of the current study.

Each relevancy matching rule can specify a data matching pattern that can be used to match the current study with a related study and/or with a clinical software application. For example, various types of pattern and/or string matching techniques such as regular expression matching may be used. The data matching pattern can be applied to the data contained in the patient model instance related to the current study.

The data matching pattern can include one or more metadata matching patterns. The metadata matching patterns can be used to match the current study with a related study and/or with a clinical software application based on the metadata associated with the current study (e.g. the metadata stored in the corresponding patient model instance).

The data matching pattern can include one or more order data matching patterns. The order data matching patterns can be used to match the current study with a related study and/or with a clinical software application based on the order data associated with the current study. For example, the order data matching pattern can be defined to identify a specified combination of HL7 tags within the order data associated with a current study. The order data matching pattern can be applied to the order data stored within the patient model instance corresponding to the current study.

The data matching pattern defined by a given relevancy matching rule can include a combination of metadata matching patterns and order data matching patterns. Optionally, the data matching pattern may be a combined matching pattern that includes a combined metadata and order data pattern.

Considering both order data received from the medical data management system(s) 304 and DICOM data received from PACS 306 can increase the relevance of the related studies and/or clinical software applications matched to the current study. In particular, incorporating the order data into the data matching pattern can ensure that the underlying clinical purpose for the current study is a factor in matching related studies and/or clinical software applications to the current study.

The order data can be used by the relevancy engine 312 to identify a clinical purpose associated with a current study being matched by the relevancy engine 312. The clinical purpose can be used to match the current study to one or more clinical applications used to process the study data. The clinical purpose may be determined inferentially based on the data contained in the patient model instance related to the current study.

The order data can also be used by the relevancy engine to identify one or more prior studies that match the current study. The matched prior studies can be retrieved by the processing system 250 to assist with the processing of the current study. The order data can be used to trigger pre-fetching of the prior studies before the current study is processed.

Optionally, the order data can be used to identify prior studies before the current study has been fully received. For example, the order data can be used to identify one or more prior studies that match the current study before some, or all, of the DICOM data associated with the current study is received by the processing system 250. The order data can also be used to trigger pre-fetching of the prior studies before the current study is fully received. This can improve processing performance as the prior studies can be fetched to the local instance cache (e.g. cache 260) even before the new current study is received by the processing system 250.

The relevancy matching rules applied by the relevancy engine 312 can be pre-defined for the medical organization 114. The pre-defined relevancy matching rules may be customized for the particular organization 114, e.g. based on the HL7 schema implemented by that organization.

Alternatively or in addition, the relevancy matching rules can be defined using machine learning techniques. For example, one or more machine learning model can be defined to implement the relevancy matching rules.

The relevancy engine 312 may implemented a routing model that can apply dynamic relevancy matching rules to determine one or more clinical applications that match a current study. The output from the routing model can be used to automatically route the current study to the matched clinical software application(s).

The routing model can receive current study data as an input. For example, the current study data can be received from the patient model instance for the corresponding patient.

The current study data can include medical image data and image metadata from the current study. This medical image data and image metadata may be defined using the DICOM format. The data defined using the DICOM format may be referred to as DICOM data.

The current study data can also include order data associated with the current study. The order data may be identified through analysis of other data received by the medical data processing system, such as HL7 data contained in HL7 messages received by the medical data processing system. The current study data input to the routing model can include order data extracted from the HL7 data received by the medical data processing system. Alternatively or in addition, the current study data input to the routing model may include all of the HL7 data from one or more HL7 messages.

The routing model can output a matched application output. The matched application output can identify one or more clinical software applications that are matched to the current study. The matched application output can indicate which clinical software application(s) the current study should be routed to for processing and/or analysis. The relevancy engine 312 can use the matched application output to automatically route the current study to the one or more matched clinical software applications.

Various different methods for training the routing model can be applied. A supervised learning method can involve a user (e.g. a clinician or technician) manually selecting the matched clinical software application(s) for a training study. The manually selected matched clinical software application(s) can be used as a label for the current study data (e.g. DICOM data, order data etc.) associated with the current study that would be used as an input to the routing model. This process can be repeated for multiple current studies.

An optimization algorithm can be applied to identify a routing pattern using positive reinforcement data (where a study is matched to a particular clinical application) and negative reinforcement data (where a study is not matched to a particular clinical application). The routing patterns identified through the optimization algorithm can subsequently be applied by the routing model to automatically match a current study with one or more clinical software applications. This may eliminate the need for a user to manually select the matched clinical software application(s).

A supervised training approach may be particularly useful because of the sometimes fluid nature of HL7 data. HL7 data can be organized using different schemas for different medical organizations. For example, the precise segment, name and field number used for order data and order-related data may vary across institutions. Accordingly, the routing patterns may differ between medical organizations. Applying the supervised training method may provide a medical organization with flexibility to train the routing model based on the existing workflows developed within that organization.

Alternatively or in addition, the routing model may be trained using unsupervised learning methods. For example, the routing model may be trained using a historical study routing dataset that identifies how prior studies were routed to matched clinical software applications. Optionally, the routing model once trained may undergo further supervision and training with user input (e.g. user approval of the matching identified by the routing model) to ensure sufficient accuracy in the routing model.

The routing model can also be updated dynamically (e.g. on an ongoing basis) based on the matching performed by the relevancy engine 312.

Referring now to FIG. 4, shown therein is an example method 400 of processing medical images. Method 400 is an example of a method for processing a plurality of medical images that can route medical image data to one of a plurality of clinical software applications using order data associated with the medical images. Method 400 may be implemented using various image processing systems, such as the example processing systems 100 and 250 described herein above.

At 410, order data can be identified by the processing unit. The order data can define an order (i.e. instructions) relating to the diagnosis, treatment or care of a particular patient or a modification to an existing order (e.g. an order update, order cancellation, order discontinuation etc.). The order data can be identified within an order message (e.g. an HL7 message or FHIR message) received by the processing unit. For example, the order data can be identified by parsing the order message as described in further detail herein below with respect to steps 520 and 530 of method 500.

The order data can include various types of data relating to an order for a patient. The order data can include a unique patient identifier (e.g. an accession number) for the corresponding patient. The unique patient identifier can be used to associate the order data with other data (e.g. current study data) related to the same patient. The unique patient identifier can also be used to associate the order data with a patient model instance for the patient. Alternatively or in addition, the order data can include other patient identification data usable to associate the order data with the patient model instance for the patient (e.g. demographic data).

The order data can include clinical request data for a patient. The clinical request data can represent data corresponding to a request or instructions relating to the diagnosis, treatment or care that is to be provided to the patient. The clinical request data can be defined by a clinician attending to the patient in some manner.

Order data can be defined using the corresponding formats associated with the application or system used to input the order. For example, the order data can be defined in an HL7 format. The order data can be received in an HL7 message that is received by the processing unit. The HL7 message may be generated in response to a user inputting the order data to an application operating on a computing device connected to the medical organization network 122. The order data may be received from various systems and devices in communication with the processing unit, such as an EMR system, a RIS system, a user device etc.

For instance, an HL7 ORM-001 message may be received by the processing unit. An HL7 ORM-001 message is typically defined as a general order message that is used to transmit information about an order. The order data message can be defined to transmit data relating to an order such as a new order (a new order message”, the cancellation of an existing order (an order cancellation message), an update to the information contained in an existing order (an order information update message), the discontinuation of an order (an order discontinuation message) and so forth.

The order data can include various different types of information relating to the corresponding instructions or order. The specific type of information contained within the order data may vary depending on the nature of the instructions or order and/or the schema used by the corresponding medical organization 114. For example, the order data may include one or more of message header data, comment data, patient identification data, patient demographic data, patient visit data, insurance data, guarantor data, patient allergy data, common order data, observation request data, diagnosis data, observation data, clinical trial identification data, billing data and so forth.

At 420, a current study can be received by the processing system. The current study can be received from a corresponding imaging device. For example, the current study may be received from an enterprise image management device. Alternatively, the current study may be received directly from an image acquisition device. The current study can include associated metadata (referred to as current study metadata) and one or more current series.

The current study metadata can include a plurality of study metadata elements. The current study metadata elements can provide information relating to various aspects of the corresponding current study. The study metadata elements can include a study instance identifier (or UID), a study date, a study time, a referring physician's name, a study identifier, a unique patient identifier (e.g. an accession number), a study description, a referenced study sequence, a referenced SOP class identifier (or UID), and a reference SOP instance identifier (or UID). Other study metadata may include an admitting diagnosis description, a patient age, and a patient weight.

The current study metadata elements may be defined manually by a user and/or automatically by components of the system 100, such as the enterprise image management device, PACS or MWL server. For example, current study metadata may be defined automatically in response to, or as part of, collection of the corresponding study (for example, the date and time(s) of a study may be automatically populated by an enterprise image management device when the study is collected).

As noted above, the current study data can include a unique patient identifier (e.g. medical record number) for the corresponding patient. The unique patient identifier can be used to associate the current study data with other data (e.g. order data) related to the same patient. The unique patient identifier can also be used to associate the current study data with the patient model instance for the patient.

Each current series can have associated current series metadata. The current series metadata can include a plurality of series metadata elements. The current series metadata elements can provide information relating to various aspects of the corresponding current series that forms part of the current study received at 420. The series metadata elements can include a modality, a series instance UID, a series number, a series date, a series time, a performing physician's name, a protocol name, a series description, an operator's name, a referenced performed procedure step sequence, a referenced SOP class UID, a referenced SOP instance UID, a request attributes sequence, a requested procedure ID, a scheduled procedure step ID, a scheduled procedure step description, a scheduled protocol code sequence, a performed procedure step ID, a performed procedure step start date, a performed procedure step start time, a performed procedure step description, a performed protocol code sequence, a comments on the performed procedure step among other metadata.

The current series metadata elements may be defined manually by a user and/or automatically by components of the system 100, such as the enterprise image management device, PACS or MWL server. For example, current series metadata may be defined automatically in response to, or as part of, collection of the corresponding series image data (for example, the date and time of a series may be automatically populated by an enterprise image management device when the series is collected).

Each current series can also include associated series image data. The series image data can define the image data collected by an image acquisition device as part of that current series. The series image data can include one or more image instances associated with the corresponding series. Each image instance may have corresponding image instance metadata associated therewith. For example, a DICOM Service Object Pair (SOP) Instance may be used to reference the image instance and its corresponding image instance metadata.

The image instance metadata can include a plurality of image instance metadata elements. The image instance metadata elements can provide information relating to various aspects of the corresponding image instance. The image instance metadata elements can include a modality manufacturer, an institution name, a station name, a manufacturer's model name, a device serial number, a software version, a private creator, an equipment UID, and a service UID, an application header sequence, an application header type, an application header ID, an application header version, workflow control flags, archive management flags for example. Additional or different image instance metadata elements may be generated, for instance based on metadata specific to the modality or image acquisition device used to generate the image instance.

The current series metadata elements may be defined manually by a user and/or automatically by components of the system 100, such as the enterprise image management device, PACS, MWL server, or image acquisition device. For example, image instance metadata may be defined automatically in response to, or as part of, collection of the corresponding image instance (for example, the date and time of an image instance may be automatically populated by an enterprise image management device when the series is collected) or when the image instance is generated (e.g. by the corresponding image acquisition device).

The current study can be sent to the processing device by various image devices, such as a PACS server or MWL server. As explained herein above, receipt of the current study can be triggered in various ways, e.g. in response to collection of the current study at an image acquisition device, or in response to parsing of order data (e.g. the order data received at 410). For example, a user may manually initiate a transmission of the current study data to the processing system. Alternatively or in addition, current study data may be transmitted to the processing system automatically upon collection by the image acquisition device (or receipt by the enterprise image management device). The current study can be pushed (or auto-forwarded) to the processing system from a PACS server or a MWL server, or alternatively from an image acquisition device.

Alternatively or in addition, the processing system may dynamically identify a current study and initiate retrieval of the current study through a pull mode of operation from the image device. The processing system can send a polling request to one or more imaging devices (e.g. a PACS server and/or an MWL server). The corresponding imaging device can response to the polling request by transmitting the current study data to the processing system.

For example, a current study can be identified based on the order data received at 410. Parsing of the order data can indicate that a new current study has been acquired. The processing system 250 can then trigger a pull operation to retrieve the new current study in response to the parsing of the order data. This may reduce resource consumption for both the PACS and the processing system 250, e.g. by reducing or eliminating the need for the processing system 250 to regularly poll for new current studies. The processing system 250 may also trigger a pull operation to retrieve related prior studies based on the parsing of the order data.

The current study (received at 420) can be identified as corresponding to the order data (received at 410) for the subject or patient. For instance, the current study metadata may be identified as corresponding to information extracted from the order data (e.g. as corresponding to a patient model instance containing the extracted order data). For instance, the order data received at 410 may be parsed to identify order data information usable to match the order data with a corresponding current study. The parsed order data information can then be used to determine that the order data corresponds to a given current study.

For instance, a unique patient identifier may be identified through parsing of the order data. The unique patent identifier can be used to identify a match between the order data and the current study. The match may be identified based on a matching unique patient identifier (e.g. a matching accession number) included in both the order data information and the current study.

Although steps 410 and 420 are shown as occurring sequentially, these steps can occur in various different orders. For instance, the order data identified at 410 may be received before or after a corresponding current study is received.

At 430, one or more relevancy matching rules can be applied to the current study. The relevancy matching rules can include clinical-application matching rules associated with the plurality of clinical software applications. The relevancy matching rules can be applied to identify a correspondence between the current study and a clinical software application that can be used to process that current study. The relevancy matching rule can include one or more matching rules relating to data associated with the current study.

The one or more relevancy matching rules can include an order data rule. The order data rule can be defined to use order data associated with the current study in order to identify a matched clinical software application. For example, order data extracted from one or more specified fields in an HL7 message can be used to identify the matched clinical software application. Examples of specified fields can include a procedure code field, a study indication field, an order notes field, a diagnosis code, and a diagnosis description field for example. These specified fields may be included in the patient model instance stored in the patient model cache 308 after being extracted from the corresponding order message.

The relevancy matching rules can be applied to the current study using the data from a patient model instance associated with the current study. The patient model instance can include the current study data (from 420) and related order data (from 410).

A relevancy matching rule can specify a set of relevant data to be identified in the patient model instance in order to match the current study with the corresponding clinical software application. For example, the relevancy matching rule can identify a set of one or more specified patient model fields within the patient model. The specified patient model fields can include fields where relevant data is expected to be found if the current study is a match according to the relevancy matching rule. The relevancy matching rule can be applied to the current study by evaluating the data contained in the specified patient model fields of the patient model instance corresponding to the current study.

The relevancy matching rule can define the specified patient model fields in various ways. The relevancy matching rule may define possible alternative specified patient model fields (e.g. where the patient model may include relevant data in multiple fields).

The relevancy matching rule can also identify, for each specified patient model field, a relevant data entry. The relevant data entry can identify the data content of the corresponding specified patient model field that indicates that the current study is a match with the corresponding clinical software application.

The relevant data entry for a given specified patient model field can be defined in various ways and alternative relevant data entries may also be defined for a given specified patient model field. For instance, various combinations of Boolean operators may be used to define the relevant data entry for a given specified patient model fields.

A specified patient model field having the corresponding relevant data entry can be identified as an application-specific relevant entry (i.e. the entry in that field is relevant to the clinical software application being matched).

The relevancy matching rule can also specify a relevant combination of specified patient model fields in order to match the current study to the clinical software applications. That is, in order to match the current study to the clinical software applications, each specified patient model field in the relevant combination of specified patient model fields should be identified as having an application-specific relevant entry. The relevant combination of specified patient model fields can be defined in various ways and alternative combinations may also be defined within the same relevancy matching rule. For instance, various combinations of Boolean operators may be used to define the relevant combination of specified patient model fields.

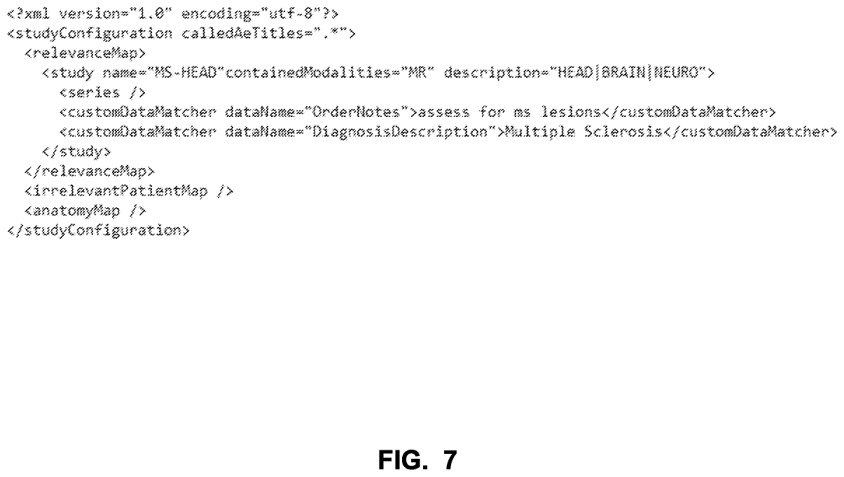

An example of a relevancy matching rule that includes a plurality of specified patient model fields and corresponding relevant data entry rules is shown in FIG. 7 described herein below.

The order data rule can also be defined to match types of order data stored for the corresponding patient (e.g. in the corresponding patient model instance stored in the patient model cache 308). The order data rule can be defined to match a pattern in various types of order data, such as the message header data, the comment data, the patient identification data, the patient demographic data, the patient visit data, the insurance data, the guarantor data, the patient allergy data, the common order data, the observation request data, the diagnosis data, the observation data, the clinical trial identification data, and the billing data for example.

The order data rule can be implemented as an order-only rule, in which only the order data associated with the current study is used to identify the matched clinical software application. For instance, the order-only rule may be defined as an order data matching pattern.

Alternatively or in addition, the order data rule can be implemented as a combined order rule, in which the order data associated with the current study is used along with additional current study data to identify the matched clinical software application. For instance, the combined order rule may be defined as a combined matching pattern that includes a combined metadata and order data pattern.

A clinical software application may have a set of relevancy rules associated with its configuration. The processing system 250 can maintain a set of rules that may be shared between the one or more clinical software applications that are configured at the processing system 250 to process incoming medical data. The relevancy rules may be defined in various ways. For instance, each clinical software application may have one or more application-specific relevancy rules that are used to match studies to that clinical software application.

An example of pseudo-code that can be used to implement an order data rule is shown in FIG. 7, described herein below.

At 440, a matching clinical software application can be identified for the current study. The matching clinical software application can be identified based on the one or more relevancy matching rules applied at 430. In particular, the matching clinical software application can be identified from amongst the plurality of clinical software applications 266 based on the order data rule applied at 430.

At 450, an assembled study set can be generated. The assembled study set can include the current study received at 420.

Optionally, the assembled study set can also include one or more matched prior studies along with the current study. The matched prior studies can be identified using one or more prior study relevancy matching rules.

The relevancy matching rules applied at 430 can also include one or more related study relevancy matching rules. Relevant prior studies may be determined by identifying one or more related study relevancy matching rules associated with the matching current study relevancy rule and applying those rules to candidate related studies. The related study relevancy matching rules may also be applied to prior study metadata associated with one or more candidate prior studies. This can allow the processing system 250 to retrieve the prior studies to assist in, or supplement, the processing of the current study. This allows image data relevant to the current study to be retrieved while non-relevant or irrelevant data is not retrieved.

The processing system 250 may transmit a request to the enterprise image management devices for one or more candidate prior studies based on the matched current study. The one or more candidate prior studies can each include prior study metadata and one or more prior series. Each of the one or more prior series can have associated prior series metadata. The processing system 250 may receive the one or more candidate prior studies from the at least one enterprise image management device of the plurality of enterprise image management devices.

Optionally, the processing system 250 may receive only metadata associated with the candidate prior studies. The metadata may be sufficient to apply the related study relevancy matching rules without requiring the transmission of associated image data.

The processing system 250 can apply a related study order data rule to the one or more candidate prior studies. A matched prior study can be identified from the one or more candidate prior studies using the related study order data rule.

Optionally, the matched prior studies may be identified using a specified subset of the related study rules determined for the matched current study. Each current study relevancy matching rule can have an associated set of related study rules. The related study rules to be applied to the candidate prior studies can be determined based on the current study relevancy matching rule used to determine that the current study is a current study.

That is, the current study may be determined to be a matched current study in response to applying a particular relevancy matching rule (e.g. because the corresponding patient model instance satisfies the requirements of that particular relevancy matching rule). That particular relevancy matching rule can have one or more associated related study rules. Those associated related study rules can then be applied to the candidate prior studies to identify any matched prior studies.

In response to identifying a matched prior study, the processing system 250 can send a query or queries to the enterprise image management devices for matched prior study data (included prior series data) corresponding to the matched prior study. The corresponding prior study data can then be received by the processing system 250. The prior study data can be included with the current study data in order to provide an assembled matched study set. The assembled matched study set can then be provided to the clinical software application(s) 266 for processing.

At 460, the assembled study can be processed using the matching clinical software application identified at 440. Processing the assembled study set can include retrieving image data corresponding to the assembled matched study set (i.e. image data for the matched current study and any matched prior studies). The image data corresponding to the assembled matched study set can then be provided to the matching clinical software application for processing.

The processing system 250 can process the assembled matched study set and the image data corresponding to the assembled matched study set using the matching clinical software application to generate a processed study. The processed study can include one or more of processed patient metadata, processed imaging data and processed order data (i.e. study metadata, study image data, or study order data that was generated or modified as a result of the processing performed by the matching clinical software application).

The processed study (generated based on the assembled matched study set) can then be provided to a medical organization user device 124. The processed study can be reviewed by a clinician at the medical organization user device 124 in order to assess a patient's condition.

Optionally, the processed study may be routed to a particular medical organization user device 124 based on the order data associated with the corresponding current study. For instance, a physician or clinician identifier associated with the order data may be used to identify the particular medical organization user device 124 (e.g. as the device 124 typically accessed by that physician or clinician).

The processed study can also be provided to one or more enterprise image management devices 128, e.g. for storage and/or further analysis. The processed study can be defined in a DICOM format to facilitate storage by the enterprise image management devices 128 and to facilitate integrating the processed study into existing image management workflows.

The matched clinical application can automatically process the assembled matched study set (i.e. without intervention by a clinician, technician or other user). That is, all of the steps of method 400 may be performed without requiring manual intervention by a user.

Referring now to FIG. 5, shown therein is an example method 500 of processing medical images using order data. Method 500 is an example of a method for identifying and parsing order data. The parsed order data can be used with order data rules to route corresponding medical image data for processing. Method 500 may be implemented using various image processing systems, such as the example processing systems 100 and 250 described herein above. The method 500 may be used to parse order data alone or in combination with a method for processing medical image data, such as method 400 for example.

At 510, the processing system 250 can receive an order message. The order message can be received in generally the same manner as described above at step 410. For instance, the order message can be an incoming HL7 message, such as an order message or an order update message.

At 520, the order message at 510 can be parsed. The order message can be parsed to identify order data that can be useful in matching a current study to a clinical software application for processing. For instance, the order data can be used to populate a patient model instance that in turn can be used to match current studies for the corresponding patients to a clinical software application. The order data to be identified in parsing the order message can be specified based on the fields defined by the patient model.

Order data fields in the order message may be identified as fields likely to contain information that is clinically relevant to an imaging test performed for a patient. Examples of clinically relevant fields can include a procedure code field, a study indication field, an order notes field, a diagnosis code field, a diagnosis description field and so forth. The specific clinically relevant data fields may be identified based on the messaging schema implemented by a given medical organization. The order messages can be parsed to identify relevant order data fields and then extract the order data from the relevant order data fields.

Order messages can often contain two different types of order data: structured order data and unstructured order data. The fields within an order message can be identified as structured order data fields (those fields containing structured order data) or unstructured order data fields (those fields containing unstructured data). The structured order data fields and unstructured order data fields for the order messages can be predefined for the order messages. That is, the structured order data fields and unstructured order data fields may be consistent across all of the order messages received by the processing system (and parsed by parser 310).

Structured order data can include a specified arrangement of data that is consistent between different order messages. Examples of structured order data include a patient name, a patient sex, a unique patient identifier (e.g. an accession code), a diagnosis code etc. The structured order data can typically be extracted directly from the corresponding order data field without further processing (i.e. the structured order data may be inherently normalized).

Unstructured order data can be defined by a user generating the order message without a strict structure impose on the data. Accordingly, the unstructured order data may not be consistent between different order messages. Examples of unstructured order data include various note and comment fields with an order message. The unstructured order data may need to be analyzed in order to provide consistent data across different patient models.

Parsing the order message can include an initial step of identifying relevant fields in the order message that contain information that can be used to populate the patient model instance. A subsequent step of parsing the order message can include extracting the data from the identified relevant fields. In some cases, extracting the data from the identified relevant fields may include normalizing the data in the fields. For instance, natural language processing may be used to normalize data that is being extracted from the identified relevant fields.

The order messages can be parsed according to a pre-defined parsing configuration file. The pre-defined parsing configuration file can define a set of rules that can be applied by the parser 310 when parsing incoming order messages. The parsing configuration file can be defined based on the HL7 schema used by a particular medical organization 114.

Optionally, the parsing configuration file can be updated dynamically as the parser 310 parses incoming order messages. For example, the parsing configuration file can be updated based on feedback relating to matching studies with corresponding clinical software applications. Order messages that contain data entries that are frequently determinative, or at least partially determinative, of a match between a current study and a clinical software application can be identified as relevant order messages. The parsing configuration file can be updated to accept or reject order messages based on whether the order message is a relevant order message or not.

For example, a machine learning model can be defined to evaluate the matching performed by the relevancy engine 312 (steps 430 and 440 of method 400). The machine learning model can be configured to identify relevant/non-relevant responses for each combination of an order message (related to a current study) and a clinical application. A confidence score can then be defined to reflect the match between the order message and the clinical application.

The confidence score can be used to determine whether the order message is a relevant order message. For instance, a relevance threshold may be defined (e.g. manually by a user or automatically by the relevancy engine). Order messages with a confidence score at or above the relevancy threshold can be identified as relevant order messages. Order messages with a confidence score below the relevancy threshold can be identified as non-relevant order message. Non-relevant order messages may be rejected by the parser 310.

The parsing of the order message can identify clinical request data associated with a particular imaging test. The clinical request data can provide an indication as to the clinical purpose underlying the imaging test. This clinical purpose can be instructive as to the type of the processing that would be desirable for the corresponding image data so that a clinician is provided with the most relevant processed data.

At 530, relevant order data can be identified within a specified data field. As noted above, the specified data field may be a data field that corresponds to a patient model field of the patient model that defines the structure of the patient model instances stored in the patient model cache 308. Data within those specified data fields may be identified as being relevant order data.

At 540, the relevant order data identified at 530 can be added to a patient model instance for the corresponding patient. The relevant order data, along with related DICOM data, can be used to populate the patient model instance for the corresponding patient. The relevant order data identified at 530 can be extracted from the order message received at 510. This extracted order data can then be added to the corresponding patient model field of the patient model instance for that corresponding patient. For instance, the extracted order data can be added to the corresponding patient model field of a patient model instance stored by the patient model cache 308.

Process 500 may be repeated for multiple order message relating to a particular patient. The patient model instance, once populated, can then be used to identify a matching clinical software application for a current study associated with that patient, for instance as described above in relation to steps 430 and 440 of method 400.

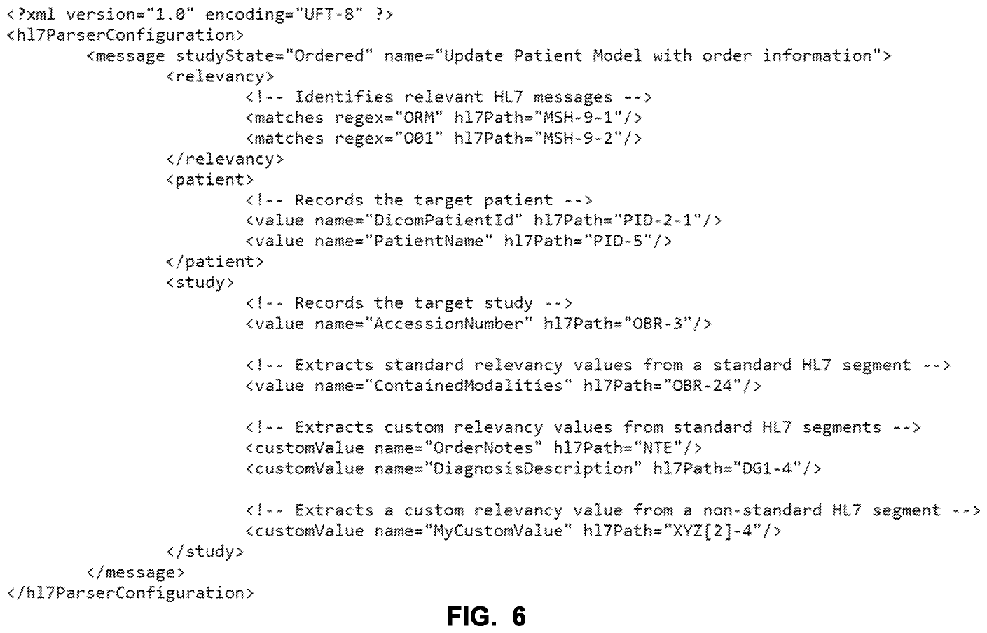

Referring now to FIG. 6, shown therein is a screenshot of pseudo-code defining an example parser configuration file. The parser configuration file can be used by various components of systems 100 and 250, such as the parser 310. The parser configuration file can define a set of rules that can be applied by the parser 310 when parsing incoming HL7 messages that can contain order data.

The values derived from parsing incoming order messages can be used to populate a patient model instance for a given patient (e.g. based on a unique patient identifier such as the accession number). This patient model instance can then be stored in the patient model cache 308 to be accessed by the relevancy engine 312. The configuration file specifies locations (e.g. message fields or segments) within a given order message from which specified types of order data can be extracted. The extracted order data can then be input to the patient model with an appropriate label or identified to facilitate processing by the relevancy engine 312.

In the example illustrated, the configuration file is defined to enable the system to parse HL7 v2.x messages. As shown in the example of FIG. 6, the configuration file can identify a plurality of specified order data fields. Each specified order data field can be defined to correspond to a specified type of order data. The specified type of order data for a given specified order data field can be defined on a global level (e.g. based on a property of the data) and/or on a model level (e.g. as corresponding to a particular subject model field of the patient model instances stored in the patient model cache 308).

Each specified order data field can be associated with a particular location within the received order message. That is, each specified order data field can be defined to correspond to a specified location within a received order message. The information contained in the order message at the specified location can then be associated with the corresponding type of order data.

In the example shown in FIG. 6, the pseudo-code <value name=“AccessionNumber”hl7Path=“OBR-3”/> relates the accession number property with the value in the third field of the first OBR segment of the HL7 message.

The pseudo-code <customValue name=“DiagnosisDescription” hl7Path=“DG1-4”/> associates the contents of the fourth field of the first DG1 segment of the received message with a patient model field defined as ‘Diagnosis Description’.

The pseudo-code <customValue name=“MyCustomValue” hl7Path=“XYZ[2]-4”/> associates the contents of the fourth field of the second XYZ segment of the received message with a patient model field defined as ‘MyCustomValue’.

The pseudo-code in the example parser configuration file defines a location for an ‘Order Notes’ field. Pseudo-code <customValue name=“OrderNotes” hl7Path=“NTE”/> associates the contents of the first NTE segment of the received message with a patient model field defined as OrderNotes’. The ‘Order Notes’ location may be a free-form (i.e. user-configurable) text field within the received order message.

In FIG. 6, the example configuration file is shown in XML, but may be prepared in any other data configuration format as is known. The configuration file may also be created using a GUI, such as via a web application form.

Referring now to FIG. 7, shown therein is a screenshot of pseudo-code defining an example relevancy matching rule. The example relevancy matching rule shown in FIG. 7 is an example of a combined order rule that can be used by the relevancy engine 312 to determine if a current study associated with a patient (i.e. a current study contained in a patient model instance stored in patient model cache 308) is relevant for processing by a particular clinical application, in this example a clinical application that is used to diagnose Multiple Sclerosis.

The example relevancy matching rule shown in FIG. 7 includes a plurality of specified patient model fields, for example the “study name” field, the “modalities” field, and the “description” field of the current study data are identified as specified patient model fields. The example relevancy matching rule shown in FIG. 7 also includes relevant data entries associated with each of the specified patient model fields relating to the current study data. As shown in FIG. 7, the relevant data entries can be defined to include alternative relevant data entries (e.g. the data entries HEADIBRAININEURO identified as alternative relevant data entries for the “description” field).

The example relevancy matching rule shown in FIG. 7 also includes an order data rule. The example order data rule shown in FIG. 7 is defined to identify the presence of magnetic-resonance studies of the patient's head (e.g. using the “study name” field, the “modalities” field, and the “description” field) that are associated with order data that includes a specified relevant combination of specified patient model fields. As shown in FIG. 7, the specified relevant combination of specified patient model fields includes a ‘Diagnosis Description’ patient model field that contains the term ‘Multiple Sclerosis’ and a patient model field ‘Order Notes’ that contains the term ‘assess for ms lesions’ from order data associated with the current study.

In various examples of the systems and methods described herein, each different clinical software application can have a separate relevancy matching rule file that can be used to determine whether a current study (contained in the patient model cache 308) is relevant for processing by that clinical software application (i.e. is matched to that clinical software application). The relevancy matching rule file for each clinical application can specify a combination of patient model fields and information contained therein that can result in the clinical application being identified as a match for that clinical software application.

The example relevancy matching rule is shown in XML, but may be prepared in any other data configuration format as is known. The relevancy matching rule may also be created using a GUI, such as via a web application form.

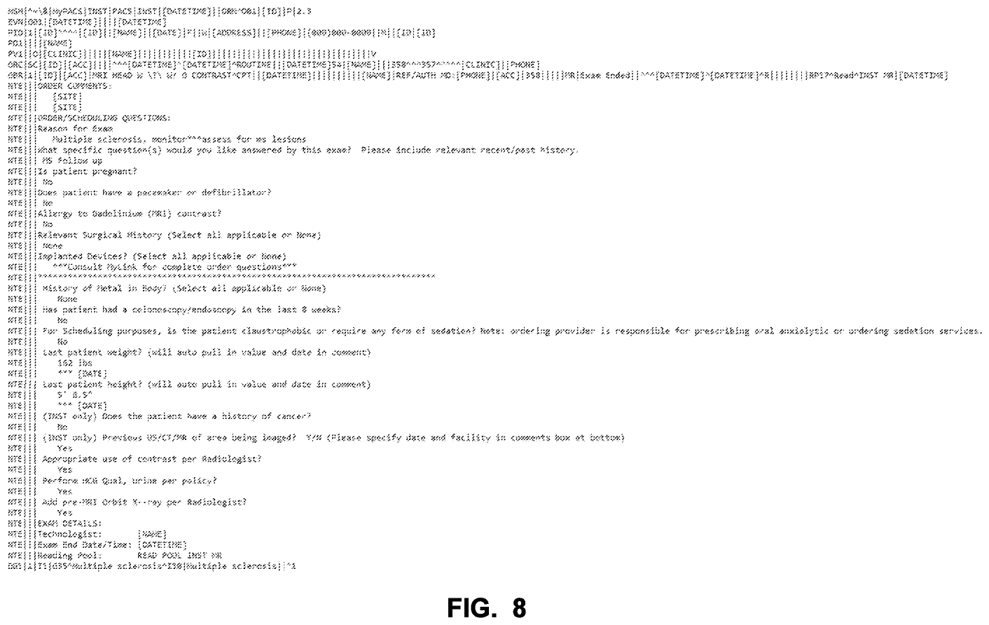

Referring now to FIG. 8, shown therein is an example of an HL7 message that may be received by a medical data processing system such as processing system 250. The example HL7 message shown in FIG. 8 is an example of an incoming order message that can be parsed by components of the processing system 250, such as the parser 308.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for processing a plurality of medical images using a plurality of clinical software applications, the method comprising:
receiving, at a processor, order data, the order data comprising clinical request data relating to a patient;
receiving, at the processor from a first imaging device, a current study corresponding to the order data for the patient, the current study comprising current study metadata and one or more current series, each of the one or more current series comprising current series metadata;
applying, at the processor, one or more relevancy matching rules associated with the plurality of clinical software applications to the current study, the one or more relevancy matching rules comprising an order data rule, wherein the order data rule is applied to the order data corresponding to the current study;
based on the applying the one or more relevancy matching rules associated with the plurality of clinical software applications:
determining, at the processor, that the current study is a matched current study; and
identifying, at the processor, a matching clinical software application in the plurality of clinical software applications based on applying the order data rule,
generating, at the processor, an assembled matched study set comprising the matched current study;
retrieving, at the processor, image data corresponding to the assembled matched study set; and
processing, at the processor the assembled matched study set and the image data corresponding to the assembled matched study set using the matching clinical software application to generate a processed study.

2. The method of claim 1, wherein the determining, at the processor, that the current study is a matched current study further comprises:
sending, from the processor to each of a plurality of enterprise image management devices, a query for one or more candidate prior studies based on the matched current study, the one or more candidate prior studies each comprising prior study metadata and one or more prior series, each of the one or more prior series comprising prior series metadata;
receiving, at the processor from at least one enterprise image management device of the plurality of enterprise image management devices, candidate prior study data relating to the one or more candidate prior studies;
determining, at the processor, that the one or more candidate prior studies are one or more matched prior studies by:
applying a related study order data rule of the one or more relevancy matching rules to the one or more candidate prior studies; and
wherein the assembled matched study set further comprises the one or more matched prior studies.

3. The method of claim 2, wherein the current study is determined to be the matched current study based on applying one or more particular matching rules of the one or more relevancy matching rules, and applying the related study order data rule comprises:
identifying a particular related study order data rule associated with the one or more particular matching rules; and
applying the related study order data rule by applying the particular related study order data rule.

4. The method of claim 1, wherein the order data is received from an EMR system.

5. The method of claim 1, wherein the order data is received from an RIS system.

6. The method of claim 1, wherein:
the order data rule defines a data matching pattern that specifies a set of relevant data from one or more data fields that must be matched in order to satisfy the order data rule.

7. The method of claim 1, further comprising:
receiving at least one order message at a parser engine;
parsing, using the parser engine at the processor, the at least one order message to identify the order data comprising the clinical request data based on at least one clinical request parsing rule.

8. The method of claim 1, wherein the current study is received at the processor directly from an image acquisition device.

9. The method of claim 1, wherein each relevancy matching rule specifies one or more corresponding patient model fields, and applying each relevancy matching rule comprises:
determining the presence or absence of relevant data within the one or more corresponding patient model fields of the patient model associated with the current study; and
determining that the current study is matched according to that relevancy matching rule in response to determining the presence of the relevant data within the one or more corresponding patient model fields.

10. The method of claim 9, wherein for at least one of the relevancy matching rules the at least one patient model field to be evaluated is selected from the group of: an order notes field, a diagnosis description field, a diagnosis code field, a procedure code field, and a study indication field.

11. The method of claim 1, wherein the processed study is provided in a DICOM format.

12. The method of claim 11, wherein the processed study comprises at least one of processed patient metadata, processed imaging data and processed order data.

13. The method of claim 1, wherein the matched clinical application automatically processes the assembled matched study set.

14. The method of claim 1, wherein the patient data is defined using a DICOM data format.

15. The method of claim 1, wherein the order data is defined using an HL7 format or a FHIR format.

16. A system for processing a plurality of medical images using a plurality of clinical software applications, the system comprising:
a memory, wherein one or more relevancy matching rules are stored in the memory, the one or more relevancy matching rules associated with the plurality of clinical software applications, the one or more relevancy matching rules comprising an order data rule;
a network device;
a processor in communication with the memory and the network device, the processor configured to:
receive, using the network device, order data, the order data comprising clinical request data relating to a patient;
receive, using the network device from a first imaging device, a current study corresponding to the order data for the patient, the current study comprising current study metadata and one or more current series, each of the one or more current series comprising current series metadata;
apply the one or more relevancy matching rules to the current study, wherein the order data rule is applied to the order data corresponding to the current study;
based on the applying the one or more relevancy matching rules:
determine that the current study is a matched current study; and
identify a matching clinical software application in the plurality of clinical software applications based on applying the order data rule;
generate an assembled matched study set comprising the matched current study;
retrieve, using the network device, image data corresponding to the assembled matched study set; and
process the assembled matched study set and the image data corresponding to the assembled matched study set using the matching clinical software application to generate a processed study.

17. The system of claim 16, wherein the processor is further configured to determine that the current study is a matched current study by:
sending, using the network device to each of a plurality of enterprise image management devices, a query for one or more candidate prior studies based on the matched current study, the one or more candidate prior studies each comprising prior study metadata and one or more prior series, each of the one or more prior series comprising prior series metadata;
receiving, using the network device from at least one enterprise image management device of the plurality of enterprise image management devices, candidate prior study data relating to the one or more candidate prior studies;
determining that the one or more candidate prior studies are one or more matched prior studies by:
applying a related study order data rule of the one or more relevancy matching rules to the one or more candidate prior studies; and
wherein the assembled matched study set is generated to include the one or more matched prior studies.

18. The system of claim 16, wherein the processor is configured to:
determine that the current study is the matched current study based on applying one or more particular matching rules of the one or more relevancy matching rules; and
apply the related study order data rule by:
identifying a particular related study order data rule associated with the one or more particular matching rules; and
applying the related study order data rule by applying the particular related study order data rule.

19. The system of claim 16, wherein the order data is received from an EMR system.

20. The system of claim 16, wherein the order data is received from an RIS system.

21. The system of claim 16, wherein:
the order data rule defines a data matching pattern that specifies a set of relevant data from one or more data fields that must be matched in order to satisfy the order data rule.

22. The system of claim 16, wherein the processor is further configured to:
receive at least one order message at a parser engine;
parse, using the parser engine, the at least one order message to identify the order data comprising the clinical request data based on at least one clinical request parsing rule.

23. The system of claim 16, wherein the current study is received using the network device directly from an image acquisition device.

24. The system of claim 16, wherein each relevancy matching rule specifies one or more corresponding patient model fields and the processor is further configured to apply each relevancy matching rules by:
determining the presence or absence of relevant data within the one or more corresponding patient model fields of the patient model associated with the current study; and
determining that the current study is matched according to that relevancy matching rule in response to determining the presence of the relevant data within the one or more corresponding patient model fields.

25. The system of claim 24, wherein for at least one of the relevancy matching rules the at least one patient model field to be evaluated is selected from the group of: an order notes field, a diagnosis description field, a diagnosis code field, a procedure code field, and a study indication field.

26. The system of claim 16, wherein the processed study is provided in a DICOM format.

27. The system of claim 16, wherein the processed study comprises at least one of processed patient metadata, processed imaging data and processed order data.

28. The system of claim 16, wherein the matched clinical application is configured to automatically process the assembled matched study set.

29. The system of claim 16, wherein the current study data is defined using a DICOM data format.

30. The system of claim 16, wherein the order data is defined using an HL7 format or a FHIR format.

31. A non-transitory computer-readable medium with instructions stored thereon for processing a plurality of medical images using a clinical software application, that when executed by a processor, performs the method of claim 1.
